(19) United States
(12) Patent Application Publication
(10) Pub. No.: US 2010/0081632 A1
(43) Pub. Date: Apr. 1, 2010

(54) High-content and high throughput assays for identification of lipid-regulating pathways, and novel therapeutic agents for lipid disorders

(75) Inventors: Donna Oksenberg, Palo Alto, CA (US); Drew Sukovich, Martinez, CA (US); Tomoe Minami, Dublin, CA (US); Jane Lamerdin, Livermore, CA (US); John K. Westwick, San Ramon, CA (US)

Correspondence Address: Isaac A. Angres;Suite 304B, 2001 Jefferson Davis Highway, Arlington, VA, 22202, US

(73) Assignee: Odyssey Thera, Inc., San Ramon, CA (US)

(21) Appl. No.: 12/382,066

(22) Filed: Mar. 6, 2009

Related U.S. Application Data
(60) Provisional application No. 61/064,462, filed on Mar. 6, 2008.

(51) Int. Cl. A61K 31/69; G01N 33/53; G01N 33/573; C40B 30/04; A61K 31/35

(52) U.S. Cl. 514/64; 435/7.1; 435/7.4; 506/9; 514/460

(57) ABSTRACT

A method of assaying protein-protein interactions associated with proteins involved in lipid pathways using a protein fragment complementation assays, said method comprising the steps of: (a) identifying protein molecules that interact with said protein associated with lipid pathways; (b) selecting a protein reporter molecule; (c) effecting fragmentation of said protein reporter molecule such that said fragmentation results in reversible loss of reporter function; (d) fusing or attaching fragments of said protein reporter molecule separately to said interacting protein molecules as defined in step (a); (e) transfecting cells with nucleic acid constructs coding for the products of step (d); (f) reassociating said reporter fragments through interactions of the protein molecules that are fused or attached to said fragments; and (g) measuring directly or Indirectly the activity of said reporter molecule resulting from the reassociation of said reporter fragments.

This application claims the priority benefit under 35 U.S.C. section 119 of U.S. Provisional, Patent Application No. 61/064,462 entitled “High-Content, High Throughput Assays For Monitoring Lipid-Regulating Pathways And Discovery Of Novel Therapeutic Agents”, filed Mar. 6, 2008, which is in its entirety herein incorporated by reference.

### FIELD OF THE INVENTION

This invention relates generally to the fields of biology, molecular biology, chemistry and biochemistry. The invention relates to novel protein complementation assays (PCA) for interactions between proteins associated with lipid regulating pathways. The invention is also directed to a large number of novel protein complementation assays (PCA) for interactions between PCSK9 (Proprotein convertase subtilisin kexin 9) and LDLR (low density lipoprotein receptor). The invention also relates to methods for constructing such assays for one or more steps. The invention can be used for functional characterization of targets and target validation, de-orphanization of receptors, high-throughput screening, high-content screening, pharmacological profiling, and other drug discovery applications.

The assays can be used directly to assess whether a compound library or a biological extract contains an agonist or antagonist of a receptor. Assay compositions are also provided. The development of such assays is shown to be straightforward, providing for a broad, flexible and biologically relevant platform for the discovery of novel drugs and natural ligands that act on the proteins directly or within pathways linked to the proteins comprising the assays. The invention is demonstrated for a broad range of proteins and for a range of assay formats.

The present invention more specifically relates to PCA expression constructs for wild type and mutant forms of PCSK9. The present invention is also directed to pharmacological drug design using PCA assays for studying PCSK9. The invention further provides methods for identifying compounds that regulate the PCSK9/HDL complex, either directly or indirectly. The instant invention further relates to PCA assays for measuring complex formation between PCSK9 and LDLR and pathways linked to that complex.

### BACKGROUND OF THE INVENTION AND DESCRIPTION OF THE PRIOR ART

Cardiovascular disease is the leading cause of death in the United States and most developed countries (US Center for Disease Control). A primary cause of cardiovascular disease is the development of atherosclerotic plaques. The link between plasma cholesterol levels and atherosclerosis, and the discovery that cholesterol-lowering drugs can forestall heart disease, rank among the seminal discoveries of modern medicine.

Atherosclerosis (or arteriosclerosis) is the term used to describe progressive narrowing and hardening of the arteries that can result in an aneurysm, thrombosis, ischemia, embolism formation or other vascular insufficiency. The disease process can occur in any systemic artery in the human body. For example, atherosclerosis in the arteries that supply the brain (e.g., the carotids and intracerebral arteries) can result in stroke. Gangrene may occur when the peripheral arteries are blocked, and coronary artery disease occurs when the arteries that supply oxygen and nutrients to the myocardium are affected. The atherosclerotic process involves lipid-induced biological changes in the arterial walls resulting in a disruption of homeostatic mechanisms that keep the fluid phase of the blood compartment separate from the vessel wall. The atheromatous plaque consists of a mixture of inflammatory and immune cells, fibrous tissue, and fatty material such as low density lipoproteins (LDL). The incidence of atherosclerosis is continuing to increase as a result of the Western diet and the growing proportion of elderly in the population. Additionally, since atherosclerosis is the primary cause of myocardial infarction, cerebral infarction, cerebral apoplexy and so forth, there remains a critical need for improved prevention and treatment.

The average American consumes about 450 mg of cholesterol per day, and produces an additional 500 to 1,000 mg in the liver and other tissues. Another source of cholesterol is the 500 to 1,000 mg of biliary cholesterol that is secreted into the intestine daily; about 50 percent is reabsorbed. The link between plasma cholesterol and the incidence of atherosclerosis and coronary heart disease is well-established. Atherosclerotic plaque inhibit blood flow, promote clot formation and can ultimately cause heart attacks, stroke and claudication.

Elevated serum cholesterol levels (>200 mg/dL) have been indicated as a major risk factor for heart disease. As a result, experts have recommended that those individuals at high risk decrease serum cholesterol levels through dietary changes, a program of physical exercise, and lifestyle changes. It is recommended that the intake of saturated fat and dietary cholesterol be strictly limited and that soluble fiber consumption be increased. Limiting the intake of saturated fat and cholesterol does not present a risk to health and nutrition. Even where saturated fat and cholesterol are severely restricted from the diet, the liver remains able to synthesize sufficient quantities of cholesterol to perform necessary bodily functions.

The regulation of cholesterol homeostasis in humans and animals involves modulation of cholesterol biosynthesis, bile acid biosynthesis, and the catabolism of the cholesterol-containing plasma lipoproteins. The liver is the main organ responsible for cholesterol biosynthesis and catabolism and, for this reason, it is a prime determinant of plasma cholesterol levels. The liver is the site of synthesis and secretion of very low density lipoproteins (VLDLs) which are subsequently metabolized to low density lipoproteins (LDLs) in the circulation. LDLs are the predominant cholesterol-carrying lipoproteins in the plasma and an increase in their concentration is correlated with increased atherosclerosis.

More recently, experts have begun to examine the individual components of the lipid profile, in addition to the total cholesterol level. While an elevated total cholesterol level is a risk factor, the levels of the various forms of cholesterol which make up total cholesterol may be more specific indicators of risk. Elevated low-density lipoprotein (LDL) is a particular cause for concern, as these loosely packed lipoproteins are more likely to lodge within the cardiovascular system, leading to the formation of atherosclerotic plaques. Low levels of high-density lipoproteins (HDL) are an additional risk factor, as HDLs serve to sequestor artery clogging cholesterol from the blood stream. A better indication of risk appears to be the ratio of total cholesteron:HDL.

Another important factor in determining cholesterol homeostasis is the absorption of cholesterol in the small intestine. On a daily basis, the average human consuming a Western diet eats 300 to 500 mg of cholesterol. In addition, 600 to 1000 mg of endogenously produced cholesterol can traverse the intestines each day. This cholesterol is a component of bile and is secreted from the liver. The process of cholesterol absorption is complex and multifaceted. The literature on cholesterol illustrates that approximately 50% of the total cholesterol within the intestinal lumen is absorbed by the cells lining the intestines (i.e., enterocytes). This cholesterol includes both diet-derived and bile- or hepatic-derived cholesterol. Much of the newly-absorbed cholesterol in the enterocytes is esterified by the enzyme acyl-CoA:cholesterol acyltransferase (ACAT). Subsequently, these cholesteryl esters are packaged along with triglycerides and other components (i.e., phospholipids, apoproteins) into another lipoprotein class, chylomicrons.

Chylomicrons are secreted by intestinal cells into the lymph where they can then be transported to the blood. Virtually all of the cholesterol absorbed in the intestines is delivered to the liver by this route. When cholesterol absorption in the intestines is reduced, by whatever means, less cholesterol is delivered to the liver. The consequence of this action is a decreased hepatic lipoprotein (VLDL) production, and an increase in the hepatic clearance of plasma cholesterol, mostly as LDL. Thus, the net effect of an inhibition of intestinal cholesterol absorption is a decrease in plasma cholesterol levels.

Elevated levels of Low Density Lipoprotein Cholesterol particles (LDLc), or so called “bad” cholesterol, are clearly associated with a high risk of heart disease. LDL receptors are plasma membrane glycoproteins that remove LDL from the plasma. A higher level of these receptors, particularly in hepatocytes, acts to decrease circulating LDLc, and thereby decrease subsequent morbidity and mortality due to atherosclerotic plaques.

A pharmacological approach to decreasing circulating LDLc entails the use of HMGCoA Reductase inhibitors, or statins. HMGCoA Reductase is a key enzyme in the cholesterol biosynthetic pathway, and its inhibition reduces circulating levels of LDLc. However, about half of the patients taking statin drugs to reduce cholesterol cannot reduce LDLc to desired levels. Also, some patients experience significant, sometimes severe side effects following statin treatment, including rhabdomyolysis and hepatotoxicity. Finally, statins induce a feedback loop that can lead to increased PCSK9 levels, counteracting their beneficial effects. Thus, despite the tremendous success with statin therapy, interest in the development of alternative or adjuvant therapies is very high.

Alternatives to statin therapy for cholesterol control are desirable. A possible approach described recently is through control of the proprotein convertase subtilisin kexin 9 (PCSK9) protein which is a member of the subtilisin serine protease family. PCSK9 interacts with LDL receptors, and thus may be a pharmacologic target for identification of cholesterol-regulating therapeutics. Support for this notion comes from several sources, notably the existence of human populations with polymorphisms in PCSK9 alleles. Individuals with specific PCSK9 variants have been found to be more, or less susceptible to atherosclerosis and cardiovascular disease (depending on the particular variant). In addition, studies using antisense oligonucleotides targeting PCSK9 in non-human primates have shown dramatic improvements in LDL cholesterol levels with minimal effects on HDL cholesterol, suggesting that inhibition of PCSK9 will be beneficial. However, specific small molecule regulators of PCSK9 have not been described.

Researchers have recently discovered a region on human chromosome 1 that segregates with autosomal dominant hypercholesterolemia (ADH) in a population of French families (1). PCSK9 was identified as the responsible gene; 2 mis-sense mutations, S172R and F216L associated with ADH were subsequently discovered. Another mutation D374Y associated with ADH (D374Y) was discovered in a Norwegian kindred (2) and a Utah pedigree (3). Additional studies indicated that PCSK9 was regulated by cholesterol (4, 5). Loss of function mutations have also been described that are associated with markedly lower plasma cholesterol levels and strong protection against coronary heart disease (CHD) (6, 7, 8 and 9).

PCSK9 is the 9^{th} member of the mammalian proprotein convertase family of serine endoproteases to be identified (10). It is synthesized as a 692 amino acid proprotein that contains a signal sequence (amino acids 1-30), a prodomain (amino acids 31-152) and a catalytic domain (153-425) (FIG. 1) (11). PCSK9 lacks a conserved P domain that is found in most other proprotein convertase family members, and is purported to be necessary for proper folding and regulation of the catalytic activity of the protein that is (12). In place of the conserved P domain, the carboxy terminus of the PCSK9 contains a cysteine- and histidine-rich region (amino acids 425-692) that shares structural homology to resistin, an adipokine linked to insulin resistance and obesity (13). The protein is synthesized as a precursor that is cleaved by autocatalytic cleavage between the prodomain and the catalytic domain (11). Like other members of the subtilisin family, the prodomain remains bound to the mature protein as it moves through the cellular secretion pathway. However, the role of the prodomain in PCSK9 function remains unknown.

By an as yet undiscovered mechanism, PCSK9 binds to the LDL receptor and decreases the number of LDL receptors expressed on the surface of cells in the liver, resulting in an increase in plasma cholesterol levels. Data from animal models closely match those observed in humans with gain and loss of function mutations. Adenoviral-mediated over-expression of PCSK9 in mice results in a low-density lipoprotein receptor knock-out phenotype characterized by an increase in plasma cholesterol levels (14). In contrast, gene deletion studies have shown that knocking out PCSK9 expression results in a decrease in plasma cholesterol levels (15). Treatment of non-human primates with RNAi targeted against PCSK9 has been shown to result in large decreases in plasma cholesterol levels (16), validating the pharmaceutical approach to regulating cholesterol levels by decreasing PCSK9 protein levels.

Several studies have indicated that the catalytic activity of the protein is not required for PCSK9 to decrease LDL receptors expressed on the cell surface (17, 18). These results suggest that standard biochemical screening methods to find catalytic activity inhibitors may fail to identify compounds that interfere with PCSK9 mediated decreases in functional LDL receptors. In addition, biochemical assays are limited to the identification of compounds that can only interfere with one possible mechanism of PCSK9 function. To date, a robust in vitro assay for PCSK9 activity has proven difficult to develop, no known substrate has been identified (save the mature protein itself), and no drug-like pharmacological inhibitors have been identified. Given the importance of this process for human disease, and the compelling human genetic and animal model validation of this protein as a potential drug target, assays that can identify regulators of PCSK9 and LDL receptor pathways are an important goal. Based on the human genetic studies, and the recent description of a 32 year old healthy female who is a compound heterozygote for PCSK9 loss of function mutations (19), antagonizing this cholesterol regulatory pathway should be relatively safe and non-toxic.

### OBJECTS OF THE INVENTION

It is an object of the present invention to provide assays that monitor the existence, cellular localization, and activity of PCSK9- and LDL-receptor-containing protein complexes;

It is also an object of the present invention to provide for additional cellular assays, based on protein complex analysis, which monitor the activity of PCSK9 as well as cellular pathways linked to PCSK9 and LDL receptors.

Still, another object of the invention is to provide a method for monitoring protein-protein interactions associated with lipid regulatory pathways.

A further object of the invention is the identification of known and novel small molecular weight pharmaceutical compositions which regulate cellular lipid levels.

A further object of the invention is the description of lipid regulatory properties inherent in certain chemical compositions previously described as protein kinase inhibitors.

Other objects and embodiments of the present invention will be discussed below. However, it is important to note that many additional embodiments of the present invention not described in this specification may nevertheless fall within the spirit and scope of the present invention and/or the claims.

### BRIEF DESCRIPTION OF THE FIGURES

FIG. 1 shows the PCSK9 structure and reported mutations. PCSK9 is synthesized as a 692 amino acid containing an N-terminal signal peptide, pro-domain, catalytic domain and a cysteine- and histidine-rich carboxy terminus. The mature protein is produced by the autocatalytic cleavage between the pro-domain and the catalytic domain. The pro-domain remains bound to the mature protein as it moves through the cellular secretion pathway.

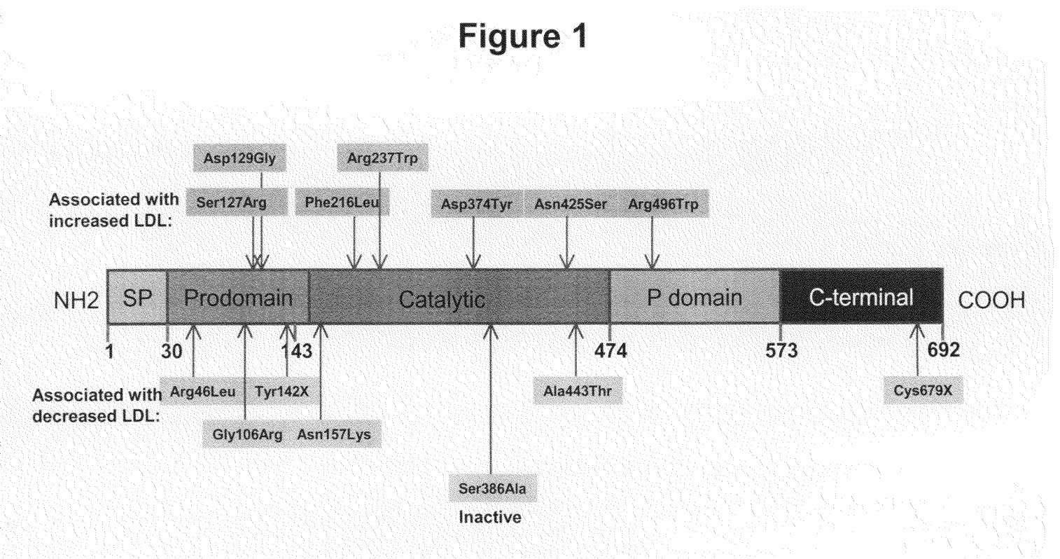

FIG. 2 illustrates the western blot analysis of HEK293T cells that were transiently transfected with PCSK9-IFP2 1 μg, (lane 3) or co-transfected with LDLR-IFP1 0.1 μg and PCSK9 0.1 μg (lane 4). HEK293T cells were transiently transfected for 24 hrs with PCSK9-IFP2 (1 ug) or co-transfected with LDLR-IFP1 (0.1 ug) and PCSK9-IFP2 (0.1 ug). A total of 10 μg cell lysate was subjected to western blot analysis with an antibody against YFP. Lanes 1 protein size marker; Lane 2: HEK293 cell lysate; Lane 3: HEK293 cells transfected with PCSK9-IFP2 construct; Lane 4: HEK293 cells transfected with PCSK9-IFP2 and LDLR-IFP1 constructs.

FIG. 3 shows. HEK293 cells that were transiently transfected with varying ratios of PCSK9-IFP2 and LDLR-IFP1 PCA constructs as indicated.

FIG. 4 illustrates the western blot analysis of different amount of concentrated culture media obtained 96 hours after HEK 293T cells were transiently transfected with wild-type PCSK9-IFP2. HEK 293T cells were transiently transfected with wild-type PCSK9-IFP2 and culture medium was collected 96 hours after transfection. Lane 1: molecular size marker; lane 2: culture medium (50 μg protein) from PCSK9-IFP2 transfected HEK293T cells; lane 3 culture medium (200 μg protein) from PCSK9-IFP2 transfected HEK 293T cells, lane 4 culture medium from untransfected HEK293T cells.

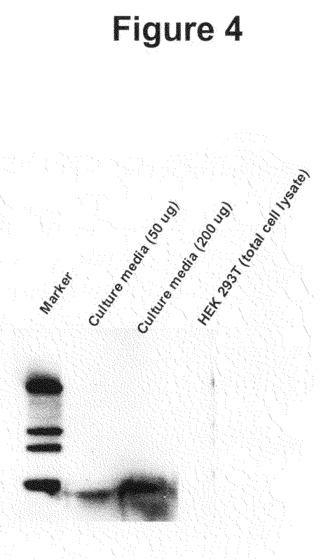

FIG. 5 illustrates the inhibition of PCSK9/LDLR interaction using small molecule non-selective proprotein convertase inhibitors.

FIG. 6 shows the effect of proton ion pumps (H+/K+ ATPase) inhibitors on PCSK9/LDLR interaction.

FIG. 7 shows the decreases of the PCA signal elicited by tyrosine kinase inhibitors, in particular non-receptor tyrosine kinase inhibitors.

FIG. 8 shows increases in LDL uptake in HepG2 cells elicited by tyrosine kinase inhibitors. The assay uses human LDL conjugated to DyLight™ 549 as a fluorescent probe for detection of LDL uptake into HepG2 cells.

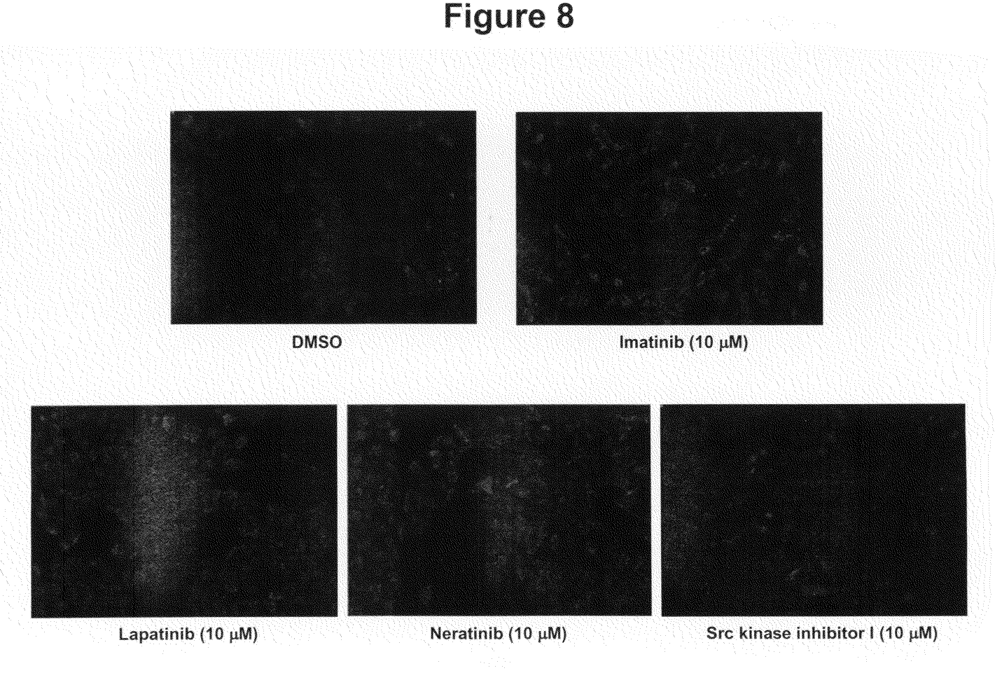

FIG. 9 shows how the LDL uptake co-localizes with LDL receptors. An LDL receptor-specific polyclonal antibody and a DyLight™ 488-conjugated secondary antibody are used for identifying the distribution of LDL receptors.

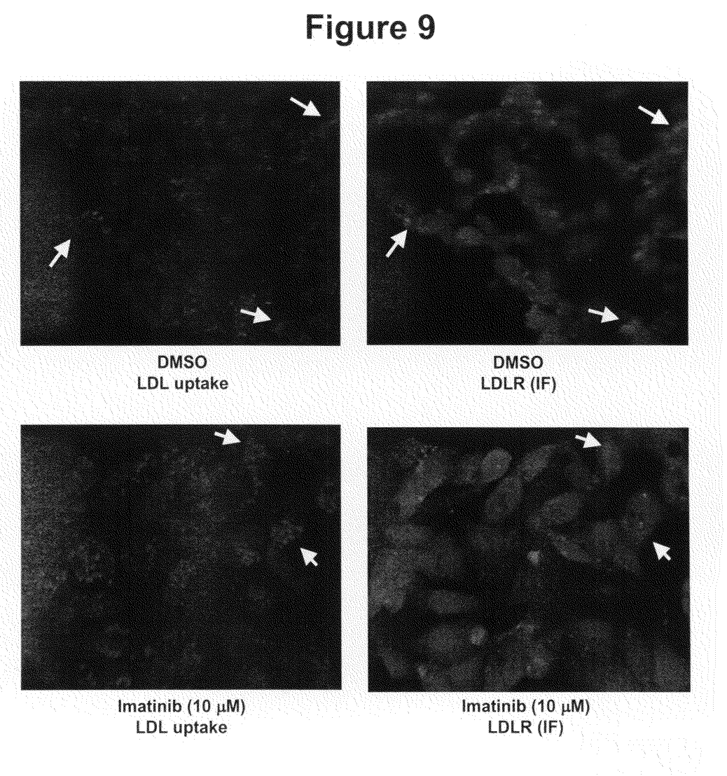

### SUMMARY OF THE INVENTION

A method of assaying protein-protein interactions associated with proteins involved in lipid pathways using a protein fragment complementation assays, said method comprising the steps of: (a) identifying protein molecules that interact with said protein associated with lipid pathways; (b) selecting a protein reporter molecule; (c) effecting fragmentation of said protein reporter molecule such that said fragmentation results in reversible loss of reporter function; (d) fusing or attaching fragments of said protein reporter molecule separately to said interacting protein molecules as defined in step (a); (e) transfecting cells with nucleic acid constructs coding for the products of step (d); (f) reassociating said reporter fragments through interactions of the protein molecules that are fused or attached to said fragments; and (g) measuring directly or indirectly the activity of said reporter molecule resulting from the reassociation of said reporter fragments.

The present invention provides a method of assaying protein-protein interactions and other cellular pathway measurements associated with the Proprotein convertase subtilisin kexin 9 (PCSK9) protein using a protein fragment complementation and additional high-content cellular assays, said method comprising the steps of: (a) identifying protein molecules that interact with said PCSK9 or LDL receptor proteins; (b) selecting a protein reporter molecule; (c) effecting fragmentation of said protein reporter molecule such that said fragmentation results in reversible loss of reporter function; (d) fusing or attaching fragments of said protein reporter molecule separately to said interacting protein molecules as defined in step (a); (e) transfecting cells with nucleic acid constructs coding for the products of step (d); (f) re-associating said reporter fragments through interactions of the protein molecules that are fused or attached to said fragments; and (g) measuring directly or indirectly the activity of said reporter molecule resulting from the re-association of said reporter fragments.

The invention also provides a method of screening a candidate drug, a compound library or a biological extract to identify activators or inhibitors of protein-protein interactions associated with the proprotein convertase subtilisin kexin 9 (PCSK9) or LDL receptor proteins using protein complementation assays, said method comprising the steps of: (a) selecting a protein reporter molecule; (b) effecting fragmentation of said protein reporter molecule such that said fragmentation results in reversible loss of reporter function; (c) fusing or attaching fragments of said protein reporter molecule separately to the PCSK9 or LDL receptor proteins and other protein molecules known to have an interaction with said PCSK9 or LDL receptor proteins; (d) transfecting cells with nucleic acid constructs coding for the products of step (c); (e) testing the effects of said candidate drug, compound library, or biological extract on the protein interaction of interest by contacting said cells as defined in step (d) with said candidate drug, compound library or biological extract; and (f) measuring and/or detecting directly or indirectly the activity resulting from the reassociation of the reporter fragments which had been fused to the interacting proteins, to identify specific agents that activate or inhibit the interaction of interest.

The invention further provides a method for identifying a drug lead that modulates the activity of protein-protein interactions between the PCSK9 protein and the LDLR protein using protein complementation assays, said method comprising the steps of: (a) assembling a collection or a library of compounds, said collection or library selected from the group consisting of candidate drugs, natural products, chemical compounds and/or biological extracts; (b) selecting a protein reporter molecule; (c) effecting fragmentation of said protein reporter molecule such that said fragmentation results in reversible loss of reporter function; (d) fusing or attaching fragments of said protein reporter molecule separately to said interacting PCSK9 protein and the LDLR protein; (e) transfecting cells with nucleic acid constructs coding for the products of step (d); (f) screening said collection or library by contacting said cells as defined in (e) with one or more test elements from said collection or library; and (g) detecting directly or indirectly the activity resulting from the re-association of the reporter fragments which had been fused to the interacting proteins, one or more properties of said assay; wherein a change in one or more properties of said assay in the presence of any of said test elements, relative to the absence of said test element, is used to identify a drug lead that modulates a PCSK9-LDLR interaction.

The invention also provides the use of known kinase inhibitors, including Glivec and other receptor and non-receptor tyrosine kinase inhibitors to treat dislipidemias. We identified these as having surprisingly robust activity on PCSK9, and more importantly activity on Lipid uptake by hepatocytes. The lipid regulatory activity of these molecules is a surprising discovery as these molecules regulate LDL and therefore represent a novel class of (potential) therapeutic agents for dislipidemias.

The invention also provides ATP-competitive kinase inhibitors, known kinase inhibitors, structures related to known kinase inhibitors, and novel molecules that inhibit protein kinases as treatment for dislipidemias. Glivec and p38 kinase inhibitors have particular effectiveness.

The invention further provides compositions of known and novel chemicals, previously described as protein kinase inhibitors, said chemicals having the property of regulating PCSK9 and LDL receptor pathways, or having the property of regulating lipid homeostasis in animal cells and whole organisms.

### DETAILED DESCRIPTION OF THE INVENTION

The present invention provides a method of assaying protein-protein interactions and other cellular pathway measurements associated with the Proprotein convertase subtilisin kexin 9 (PCSK9) protein using a protein fragment complementation (PCA) and/or other additional high-content cellular assays, said method comprising, the steps of: (a) identifying protein molecules that interact with said PCSK9 or LDL receptor proteins; (b) selecting a protein reporter molecule; (c) effecting fragmentation of said protein reporter molecule such that said fragmentation results in reversible loss of reporter function; (d) fusing or attaching fragments of said protein reporter molecule separately to said interacting protein molecules as defined in step (a); (e) transfecting cells with nucleic acid constructs coding for the products of step (d); (f) re-associating said reporter fragments through interactions of the protein molecules that are fused or attached to said fragments; and (g) measuring directly or indirectly the activity of said reporter molecule resulting from the re-association of said reporter fragments.

PCA represents a particularly useful method for measurements of the association, dissociation or localization of protein-protein complexes within the cell. PCA enables the determination and quantitation of the amount and subcellular location of protein-protein complexes in living cells.

With PCA, proteins are expressed as fusions to engineered polypeptide fragments, where the polypeptide fragments themselves (a) are not fluorescent or luminescent moieties; (b) are not naturally-occurring; and (c) are generated by fragmentation of a reporter. Michnick et al. (U.S. Pat. No. 6,270,964) teaches that any reporter protein of interest can be used for PCA, including any of the reporters described above. Thus, reporters suitable for PCA include, but are not limited to, any of a number of enzymes and fluorescent, luminescent, or phosphorescent proteins. Small monomeric proteins are preferred for PCA, including monomeric enzymes and monomeric fluorescent proteins, resulting in small (˜150 amino acid) fragments. Since any reporter protein can be fragmented using the principles established by Michnick et al., the assays of the present invention can be tailored to the particular demands of the cell type, target, signaling process, and instrumentation of choice. Finally, the ability to choose among a wide range of reporter fragments enables the construction of fluorescent, luminescent, phosphorescent, or otherwise detectable signals; and the choice of high-content or high-throughput assay formats.

In a preferred embodiment, the invention uses gene(s) encoding specific proteins of interest associated with lipid regulating pathways; preferably as characterized full-length cDNA(s). The methodology is not limited, however, to full-length clones as partial cDNAs or protein domains can also be employed. The cDNAs, tagged with a reporter or reporter fragment allowing the measurement of a protein-protein interaction, are inserted into a suitable expression vector and the fusion proteins are expressed in a cell of interest. However, endogenous cellular genes can be used by tagging the genome with reporters or reporter fragments, for example by non-homologous recombination. In the latter case, the native proteins are expressed along with the reporter tags of choice enabling the detection of native protein-protein complexes.

The instant invention requires a method for measuring protein-protein interactions and/or an equivalent, high-content assay method for a pathway sentinel. In a preferred embodiment, protein interactions associated with lipid pathways are measured within a cell. Such methods may include, but are not limited to, FRET, BRET, two-hybrid or three-hybrid methods, enzyme subunit complementation, and protein-fragment complementation (PCA) methods. In alternative embodiments, the interactions are measured in tissue sections, cell lysates or cell extracts or biological extracts. In the latter cases, a wide variety of analytical methods for the measurement of protein-protein complexes can be employed, for example, immunohistochemistry; western blotting; immunoprecipitation followed by two-dimensional gel electrophoresis; mass spectroscopy; ligand binding; quantum dots or other probes; or other biochemical methods for quantifying the specific protein-protein complexes. Such methods are well known to those skilled in the art. It should be emphasized that it is not necessary to apply a single technology to the measurement of the different protein-protein interactions. Any number or type of quantitative assays can be combined for use in conjunction with the present invention.

Enzyme-fragment complementation and protein-fragment complementation methods are preferred embodiments for this invention. These methods enable the quantification and subcellular localization of protein-protein complexes in living cells. With enzyme fragment complementation, proteins are expressed as fusions to enzyme subunits, such as the naturally-occurring or mutant alpha/beta subunits of β-galactosidase. With PCA, proteins are expressed as fusions to synthetic polypeptide fragments, where the polypeptide fragments themselves (a) are not fluorescent or luminescent moieties; (b) are not naturally-occurring; and (c) are generated by fragmentation of a reporter. Michnick et al. (U.S. Pat. No. 6,270,964) taught that any reporter protein of interest can be used in PCA, including any of the reporters described above. Thus, reporters suitable for PCA include, but are not limited to, any of a number of enzymes and fluorescent, luminescent, or phosphorescent proteins. Small monomeric proteins are preferred for PCA, including monomeric enzymes and monomeric fluorescent proteins, resulting in small (about 150 amino acid) fragments. Since any reporter protein can be fragmented using the principles established by Michnick et al., assays can be tailored to the particular demands of the cell type, target, signaling process, and instrumentation of choice. Finally, the ability to choose among a wide range of reporter fragments enables the construction of fluorescent, luminescent, phosphorescent, or otherwise detectable signals; and the choice of high-content or high-throughput assay formats.

As we have shown previously, polypeptide fragments engineered for PCA are not individually fluorescent or luminescent. This feature of PCA distinguishes it from other inventions that involve tagging proteins with fluorescent molecules or luminophores, such as U.S. Pat. No. 6,518,021 (Thastrup et al.) in which proteins are tagged with GFP or other luminophores. A PCA fragment is not a luminophore and does not enable monitoring of the redistribution of an individual protein. In contrast, what is measured with PCA is the formation of a complex between two proteins.

The present invention is not limited to the type of cell, biological fluid or extract chosen for the analysis. The cell type can be a mammalian cell, a human cell, bacteria, yeast, plant, fungus, or any other cell type of interest. The cell can also be a cell line, or a primary cell, such as a hepatocyte. The cell can be a component of an intact tissue or animal, or in the whole body, such as in an explant or xenograft; or can be isolated from a biological fluid or organ. For example, the present invention can be used in bacteria to identify antibacterial agents that block key pathways; in fungal cells to identify antifungal agents that block key pathways. The present invention can be used in mammalian or human cells to identify agents that block disease-related pathways and do not have off-pathway or adverse effects. The present invention can be used in conjunction with drug discovery for any disease of interest including cancer, diabetes, cardiovascular disease, inflammation, neurodegenerative diseases, and other chronic or acute diseases afflicting mankind.

The present invention can be used in live cells or tissues in any milieu, context or system. This includes cells in culture, organs in culture, and in live organisms. For example, this invention can be used in model organisms such as Drosophila or zebrafish. This invention can also be used in nude mice, for example, human cells expressing labeled proteins—such as with “PCA inside”—can be implanted as xenografts in nude mice, and a drug or other compound administered to the mouse. Cells can then be re-extracted from the implant or the entire mouse can be imaged using live animal imaging systems such as those provided by Xenogen (Alameda, Calif.). In addition, this invention can be used in transgenic animals in which the protein fusions representing the protein-protein interactions to be analyzed are resident in the genome of the transgenic animal.

The assay of the present invention may be a high-content assay format, or a high-throughput assay may be used in many if not all cases. In the case of an increase or decrease in the amount of a protein-protein complex in response to a chemical agent or drug, the bulk fluorescent or luminescent signal can be quantified. In the event of a shift in the subcellular location of a protein-protein complex in response to drug, individual cells are imaged and the signal emanating from the protein-protein complex, and its sub-cellular location, is detected. Multiple examples of these events: are provided herein. Some methods and reporters will be better suited to different situations. With PCA, a choice of reporters enables the quantification and localization of protein-protein complexes. Particular reporters may be more or less optimal for different cell types and for different protein-protein complexes.

It will be appreciated by one skilled in the art that, in many cases, the amount of a protein-protein complex will increase or decrease as a consequence of an increase or decrease in the amounts of the individual proteins in the complex. Similarly, the subcellular location of a protein-protein complex may change as a consequence of a shift in the subcellular location of the individual proteins in the complex. In such cases, either the complex or the individual components of the complex can be assessed and the results will be equivalent. High-content assays for individual pathway sentinels (proteins) can be constructed by tagging the proteins with a fluorophore or luminophore, such as with a green fluorescent protein (GFP) that is operably linked to the protein of interest; or by newer, self-tagging methods including SNAP tags and Halo-tags (Invitrogen, BioRad); or by applying immunofluorescence methods, which are well known to those skilled in the art of cell biology, using protein-specific or modification-specific antibodies provided by Cell Signaling Technologies, Becton Dickinson, and many other suppliers. Such methods and reagents can be used in conjunction with the protein-protein interactions provided herein. In particular, individual proteins associated with lipid pathways may be used to construct high-content assays for pharmacological profiling according to this invention.

The present invention also provides strategies and methods for detecting the effects of test compounds on modulable lipid pathways in cells. The pathway modulation strategy can be applied to pharmacological profiling in conjunction with any cell type and with any measurable parameter or assay format. Whereas test compounds may not have significant effect under basal conditions, their effects can be detected by treating a cell with the test compound and then with a pathway modulator. This strategy improves the sensitivity of the invention. For example, in some cases a test compound may have no effect under basal conditions but may have a pronounced effect under conditions where a pathway is either activated or suppressed. Any number of cellular pathways can be activated or suppressed by known modulators, which can be used to improve the sensitivity of pharmacological profiling.

The methods and assays provided herein may be performed in multiwell formats, in microtiter plates, in multispot formats, or in arrays, allowing flexibility in assay formatting and miniaturization. The choices of assay formats and detection modes are determined by the biology of the process and the functions of the proteins within the complex being analyzed. It should be noted that in either case the compositions that are the subject of the present invention can be read with any instrument that is suitable for detection of the signal that is generated by the chosen reporter. Luminescent, fluorescent or bioluminescent signals are easily detected and quantified with any one of a variety of automated and/or high-throughput instrumentation systems including fluorescence multi-well plate readers, fluorescence activated cell sorters (FACS) and automated cell-based imaging systems that provide spatial resolution of the signal. A variety of instrumentation systems have been developed to automate HCS including the automated fluorescence imaging and automated microscopy systems developed by Cellomics, Amersham, TTP, Q3DM (Beckman Coulter), Evotec, Universal Imaging (Molecular Devices) and Zeiss. Fluorescence recovery after photobleaching (FRAP) and time lapse fluorescence microscopy have also been used to study protein mobility in living cells. The present invention can also be used in conjunction with the methods described in U.S. Pat. No. 5,989,835 and U.S. Pat. No. 6,544,790.

The present invention provides a strategy to monitor the activity of PCSK9 and LDL receptor pathways, and for identification of diagnostic and therapeutic agents related to these processes. We describe methods for identifying small molecules or biologicals that disrupt pathways leading to PCSK9 and LDL receptors, including molecules that directly regulate binding of PCSK9 to the LDL receptor. A specific embodiment is in the form of a PCSK9/LDLR protein complementation assay (PCA), however this strategy includes any assay technology that monitors activity of pathways leading to regulation of PCSK9 or LDL receptors, as well as assays directly reporting on PCSK9 and LDLR interactions. These assays include but are not limited to PCA, Fluorescence resonance energy transfer (FRET), Bioluminescence resonance energy transfer (BRET), Homogenous time resolved fluorescence (HTRF), Scintillation proximity assay (SPA) Fluorescence polarization (FP), and biochemical or cell-based analysis of pathways or post-translational modification of PCSK9 and LDL receptors. The advantage of this strategy over a traditional enzyme-based biochemical assay is the flexibility it affords to identify inhibitors with a wide range of differing mechanisms of action (MOA), not solely inhibitors of catalytic activity and not limited to molecules directly binding to the assay proteins. The PCSK9/LDLR PCA or other protein-complex based assays mentioned above can be used to screen compound libraries of existing and off-patent drugs to identify lead compounds with well-known safety and pharmacokinetic profiles or can serve as the basis for a large HTS campaign to identify novel compounds suitable for medicinal chemistry efforts focused on developing a potent and selective pathway and PCSK9 antagonists. Compounds discovered using these methods are predicted to regulate LDL uptake by cells in vivo.

At its basic level, fragment complementation is a general and flexible strategy that allows measurement of the association and dissociation of protein-protein complexes in intact, living cells. In particular, PCA has unique features that make it an important tool in drug discovery:

1. Molecular interactions are detected directly, not through secondary events such as transcription activation or calcium release.

2. Tagging of proteins with large molecules, such as intact, fluorescent proteins, is not required.

3. With in vivo PCAs, proteins are expressed in the relevant cellular context, reflecting the native state of the protein with the correct post-translational modifications and in the presence of intrinsic cellular proteins that are necessary, directly or indirectly, in controlling the protein-protein interactions that are being measured by the PCA.

4. PCA allows a variety of reporters to be used, enabling assay design specific for any instrument platform, automation setup, cell type, and desired assay format. Reporters suitable for PCA include fluorescent proteins (GFP, YFP, CFP, BFP, RFP and variants thereof), photoproteins (aequorin or obelin); various enzymes including luciferases, β-lactamase, dihydrofolate reductase, beta-galactosidase, tyrosinase, neomycin or hygromycin phosphotransferase, and a wide range of other enzymes.

5. Depending upon the choice of reporter, either high-content or high-throughput assays can be constructed with PCA, allowing flexibility in assay design depending on the specific target and the way in which it responds to agonist or antagonist in the cellular context.

6. With high-content PCAs, the sub-cellular location of protein-protein complexes can be determined, whether in the membrane, cytoplasm, nucleus or other subcellular compartment; and the movement of protein-protein complexes can be visualized in response to a stimulus or inhibitor.

7. With high-throughput PCAs, the assays are quantitative and can be performed either by flow cytometry or in multi-well, microtiter plates using standard fluorescence microplate readers.

8. PCA can be used to ‘map’ proteins into signaling pathways and validate novel targets by detecting the interactions that a particular protein makes with other proteins in the context of a mammalian cell, and then determining whether the protein-protein complex can be modulated in response to an agonist, antagonist or inhibitor

Table 1 shows examples of suitable reporters that can be used with the present invention.

TABLE 1
Examples of reporters suitable for the present invention

| Protein | Nature of Signal | Reference |
|---|---|---|
| equorin monomeric calcium | Luminescence, requires cell permeable | Ungrin et. al. (1999) An automated aequorin luminescence - |
| activated photoprotein | coelenterazine luciferin and calcium | based functional calcium assay for G-protein-coupled |
| | | receptors, Anal Biochem. 272, 34-42; Rizzuto et. al. (1992) |
| | | Rapid changes of mitochondrial calcium revealed by |
| | | specifically targeted recombinant aequorin, Nature 358 |
| | | (6384): 325-327 |
| AsFP499 and related fluorescent | Fluorescence | Weidenmann et al. (2000) Cracks in the beta -can: |
| proteins from the sea anemone | | fluorescent proteins from anemonia Sulcata Proc. Natl. |
| Anemonia sulcata | | Acad. Sci. USA 97 (26): 14091-14096 |
| Beta-galactosidase | Fluorescence | Rossi, et al. (1997) Monitoring protein -protein interactions |
| | | in intact eukaryotic cells by beta -galactosidase |
| | | complementation. Proc Natl Acad Sci USA 94: 8405-8410. |
| Beta-lactamase | Fluorescence, CCF2/AM or other cell- | Michnick et. al. (2002) Nature Biotechnology 20: 619-622 |
| | permeable cephalosporin substrate | |
| Blue fluorescent proteins, BFPs | Fluorescence | Pavlakis et. al. Mutant Aequorea victorea fluorescent |
| | | proteins having increased cellular fluorescence, |
| | | U.S. Pat. No. 6,027,881 |
| “Citrine” a novel engineered | Fluorescence | Griesbeck et. al. (2001) Reducing the environmental |
| version of YFP | | sensitivity of yellow fluorescent protein. J. Biol Chem., 31: |
| | | 29188-29194 |
| Cyan fluorescent protein: ECFP | Fluorescence | Zhang et al. (2002) Creating new fluorescent probes for cell |
| and enhanced GFP and YFP: | | biology, Nature Reviews Mol. Cell Biology 3:, 906-918; |
| EGFP, EYFP | | Tsien (1998) Annu. Rev. Biochem. 67: 509-544. |
| Dihydrofolate reductase (DHFR) | Fluorescence, binding of fluorophore - | Remy & Michnick (2001). Visualization of Biochemical |
| | methotrexate to reconstituted DHFR | Networks in Living Cells. Proc Natl Acad Sci USA, 98: |
| | | 7678-7683. |
| DsRed a tetrameric red fluorescent | Fluorescence | Matz et al. (1999) Fluorescent proteins from |
| protein from discosoma coral | | nonbioluminescent anthozoa species. Nature Biotechnology, |
| | | 17 (10): 969-973 |
| EqFP611 a red fluorescent protein | Fluorescence | Wiedenmann et al. (2002) A far-red fluorescent protein with |
| from the sea anemone Entacmaea | | fast maturation and reduced oligomerization tendency from |
| quadricolor | | Entacmaea quadricolor. Proc. Natl. Acad. Sci. USA 99(18): |
| | | 11646-11651 |
| Firefly luciferase | Luminescence, requires D luciferin | Rutter et al. (1995) Involvement of MAP kinase in insulin |
| | | signaling revealed by non-invasive imaging of luciferase |
| | | gene expression in living cells, Current Biology 5 (8): |
| | | 890-899; De Wet et. al. (1985) Proc. Natl. Acad. Sci., USA 82: |
| | | 7870-7873; de Wet et. al. (1986) Methods in Enzymology, |
| | | 133, 3; U.S. Pat. No. 4,968,613. |
| GFP | Fluorescence | Remy et al. (2000) Protein interactions and |
| | | Library screening with protein fragment |
| | | complementation strategies, in: Protein -protein |
| | | interactions: a molecular cloning manual. |
| | | Cold Spring Harbor Laboratory Press. Chapter 25, |
| | | 449-475; and U.S. Pat. No. 6,270,964 |
| “Kaede” a new fluorescent protein | Fluorescence; green to red | Ando et al. (2002) An optical marker based on the uv - |
| isolated from coral | photoconversion | induced green-red photoconversion of a fluorescent protein, |
| | | Proc. Natl. Acad. Sci. USA 99 (20): 12651-12656 |
| m-RFP monomeric red | Fluorescence | Campbell et al. (2002) A monomeric red fluorescent protein. |
| fluorescent protein derived by | | Proc. Natl. Acad. Sci. USA 99 (12): 7877-7882 |
| engineering DsRed. | | |
| Obelin a 22 kd monomeric | Calcium activated photoprotein also | Campbell et al. (1988) Formation of the calcium activated |
| calcium activated photoprotein | requires coelenterazine luciferin | photoprotein obelin from apo -obelin and mRNA in human |
| | | neutrophils, Biochem J. 252 (1): 143-149 |
| PA-GFP a new mutant of YFP | Fluorescence; photoactivatable | Patterson et al. (2002) A photoactivatable GFP for selective |
| | | labeling of proteins and cells. Science 297: 1873-1877. |
| Recombinant monomeric | Fluorescence | Such enzymes can produced either by protein engineering of |
| glucuronidases/glycosidases | | the subunit interface of existing symmetrical multimeric |
| | | enzymes or suitable naturally occurring monomeric glycosyl |
| | | hydrolases and detected using cell permeable fluorescent |
| | | substrates such as e.g. the lipophilic substrate: ImaGene |
| | | Green C12 FDGlcU available from Molecular Probes; |
| | | Catalog number I-2908 |
| Reef coral Anthozoan derived | Fluorescence | Labas et al. (2002) Diversity and evolution of the green |
| GFPs | | fluorescent protein family, Proc. Natl. Acad. Sci., USA |
| | | 99(7): 4256-4262; Matz et al. (1999) Fluorescent proteins |
| | | from nonbioluminescent anthozoa species. Nature |
| | | Biotechnology 17 (10): 969-973. |
| Renilla and Ptilosarcus Green | Fluorescence | Luciferases, fluorescent proteins, nucleic acids encoding the |
| fluorescent proteins | | luciferases and fluorescent proteins and the use thereof in |
| | | diagnostics, high throughput screening and novelty items. |
| | | U.S. Pat. No. 6,436,682 B1, Aug. 20, 2002 assigned to |
| | | Prolume, Ltd. |
| Renilla luciferase. monomeric | Luminescence. Renilla luciferase | Baumik et al. (2002) Optical imaging of renilla luciferase |
| luminescent photoprotein and | requires cell-permeable coelenterazine | reporter gene expression in living mice, Proc. Natl. Acad. |
| Firefly luciferase | luciferin. Firefly luciferase requires | Sci., USA 99 (1): 377-382; Lorenz et al. (1991) Isolation |
| | D-luciferin. | and expression of a cDNA encoding renilla reniformis |
| | | luciferase, Proc. Natl. Acad. Sci., USA 88: 4438-4442. |
| “Venus” and super-enhanced YFP | Fluorescence | Nagai et al. (2002) A variant of yellow fluorescent protein |
| (SEYFP) | | with fast and efficient maturation for cell -biological |
| | | applications. Nature Biotechnology 20: 87-90 |
| Renilla mulleri, Gaussia and | Luminescence | Luciferases, fluorescent proteins, nucleic acids encoding the |
| Pleuromma luciferases | | luciferases and fluorescent proteins and the use thereof in |
| | | diagnostics, high throughput screening and novelty items. |
| | | U.S. Pat. No. 6,436,682 B1, Aug. 20, 2002 |

In one of the embodiments of the invention, the cell-based assay consists of transfected cDNAs encoding full-length PCSK9 and LDL receptor, each sequence linked in-frame to rationally designed fragments of a variant of green fluorescent protein. These two plasmid constructs were co-expressed in human HEK cells plated in 384-well poly-lysine coated plates. After 24-48 hours of incubation, drugs (or vehicle controls) were added to the media, and the existence and localization of PCSK9/LDL receptor complexes was quantified on an Opera automated confocal fluorescence microscopy platform (Perkin Elmer). Images were subjected to automated image analysis, and results quantified and subjected to statistical analysis.

The assay of the invention and other related assay technologies that measures complex formation between PCSK9 and LDLR (BRET, FRET, HTRF, SPA and FP) can be used for a number of different applications, including but not limited to:

1. Screening compounds libraries—Screening libraries of known or unknown compounds to discover inhibitors of PCSK9.

2. Determining the mechanism of action of PCSK9—Compounds that inhibit the interaction between PCSK9 and LDLR can be tested using the mutant forms of PCSK9 to determine if they inhibit a specific segment of PCSK9/LDLR degradation pathway.

3. Functional mutation characterization—Mutations in PCSK9 that have no known function in humans or have not been examined for its effects in a human population can be tested to determine their intracellular distribution and interaction with the LDLR and therefore be associated with hyper- or hypocholesterolemia. This can be used as a potential screen for filtering patients into the proper clinical trials for lipid lowering therapies.

4. Identification of the protein substrates of PCSK9-Over-expression of PCSK9 in many cell types results in the efficient degradation of the LDLR. In CHO cells, that express abundant LDLR, no degradation via the PCSK9 pathway is observed. These results have suggested that other factors are needed to interact with PCSK9 to degrade the LDLR. A PCA-based functional cDNA library screen can be performed to identify protein substrates and gain a further understanding of the mechanism for LDLR degradation.

5. The invention can be applied to other apoE binding receptors—PCSK9 can also degrade the VLDLR and APOER2 and this assay may serve as a surrogate in vitro assay to find inhibitors of these pathways. This includes other ApoE binding receptor family members like the LDL receptor related protein 1 (LRP1) that interact with a number of similar protein partners.

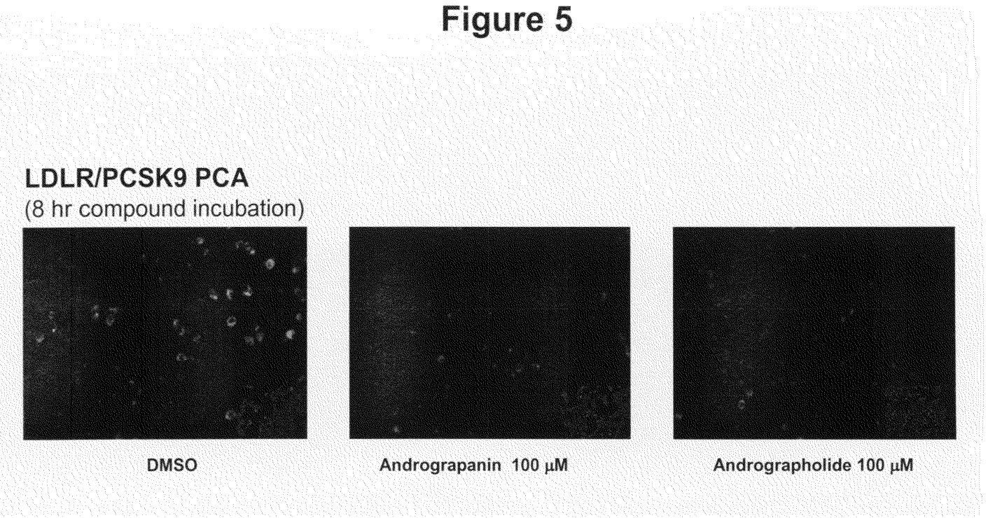

6. Profiling of ligand binding (or any soluble protein) to cognate receptor or binding protein in a large panel of cell-based assays—Our PCSK9/LDLR PCA system can be expanded for use as a large scale cell-based ligand binding system that can evaluate the binding of a panel of ligands (or any secreted, soluble protein like PCSK9) with their cognate signaling receptor (or protein binding partner) in response to drug or RNAi treatment. This can be performed with 2 different cell lines each expressing an individual PCA protein partner (i.e. receptor/ligand pair) or a single cell line expressing one PCA protein partner and the other PCA partner added exogenously (as a purified component or cell supernatant). For example using the PCSK9/LDLR protein pair we can express the LDLR in HEK293 cells and add cell supernatant from cells expressing the PCSK9 PCA construct since this protein is secreted (FIG. 5). This interaction can be measured using the same imaging system used for monitoring a co-transfected PCA pair in a single cell type. This profiling can be applied to the identification of novel inhibitors of specific signaling pathways or used to determine off-target effects of potential drug development candidates.

7. Identification of PCSK9 dimerization inhibitors—PCSK9 dimerization has been shown to be associated with its LDLR-degrading activity (22). The present invention can be used to measure PCSK9 dimerization in a screening assay to identify small molecular or biologic inhibitors of protein dimerization. The inhibition of dimerization will result in a decrease in circulating PCSK9 homodimers, an increase in LDL receptors expressed in the liver and lower plasma cholesterol levels (22).

8. Identification of compounds that stabilize the PCSK9/HDL interaction—HDL may serve as a plasma “sink” for PCSK9 preventing the binding to and degradation of the LDLR. Based on current theory this will lead to an increase in LDL receptors in the liver and lower plasma cholesterol levels (22). The functional sequestration of PCSK9 by HDL can be regulated by the stabilization or increased binding between HDL and PCSK9. The PCA assay system described here can be used to screen for compounds that increase complex formation between PCSK9 and apolipoprotein A, the major lipoprotein responsible for transport of HDL in serum. Compounds that increase complex formation can be identified by an increase in signal in the PCA.

The invention also describes a novel strategy for identification of PCSK9/LDL receptor regulators. Drugs identified using these assays were also found to also regulate cholesterol uptake by hepatocytes and other cell types. In the present invention, we have also identified specific drugs, including known drugs such as Imatinib, that regulate PCSK9/LDL receptor complexes and that affect lipid regulation. To our knowledge, the dramatic effect of these drugs, and several known kinase inhibitors in particular, on LDL uptake has not been previously reported. These drugs and analogs or variants of these drugs may have utility in therapeutic settings such as hypercholesterolemia and atherosclerosis. The purported molecular targets of these compounds are known, and targeting of these proteins may represent novel strategies for cholesterol regulation.

It is noteworthy of the invention that a number of the molecules regulating PCSK9 and lipid uptake are kinase inhibitors, in particular receptor tyrosine kinase inhibitors and receptor-associated kinases (such as c-Src, PI3-Kinase, Abl). This suggests a general but previously unappreciated role for these kinases in lipid homeostasis. The primary role of receptor tyrosine kinases is control of cellular growth and differentiation, but high levels of these kinases also exist in differentiated and non-dividing cells. Pathways downstream from these kinases control diverse cellular functions. One pathway downstream from these kinases controls Ras family GTPase activity, and effector kinases such as ROCKs and p21-activated kinases (PAKs). These kinases regulate the actin cytoskeleton, and may thereby regulate the transport of LDL receptor-lipid containing vesicles.

Regardless of the mechanism, the general strategy described in this invention was able to identify surprising and potentially valuable activities of well known drugs. The effects on the PCA assay and the lipid uptake assay occur at the same compound concentrations, validating the use of the PCSK9 PCA assay as a strategy for identification of drugs and drug candidates that regulate lipid uptake and metabolism. We predict, therefore, that this strategy can be used to identify additional novel therapeutic agents for these and other conditions related to cholesterol levels and lipid homeostasis.

### EXAMPLES

### Methods

### Example 1

### PCA Expression Constructs

The wild type coding sequence of PCSK9 was amplified by PCR from a human cDNA encoding PCSK9 (Seq I.D. No. 1; obtained from OriGene) using the following primers: forward primer 5′-ATA AGA ATG CGG CCG CAC CAT GGG CAC CGT CAG CTC CAG GCG (SEQ I.D No. 3) and reverse primer 5′-GGC GCG CCC CTG GAG CTC CTG GGA GGC CTG C(SEQ I.D No. 4). The 5′-end of the forward and reverse primers contained Not I or Asc I restriction enzyme sites, respectively, which were used to insert the coding sequence of PCSK9 in-frame with the N-terminus of the IFP2 reporter fragment via a 10 amino acid flexible linker in the mammalian expression vector pcDNA3.

The nucleotide sequence for PCSK9 (SEQ I.D. No. 1) is as follows:

atgggcaccg tcagctccag gcggtcctgg tggccgctgc
cactgctgct gctgctgctg ctgctcctgg gtcccgcggg
cgcccgtgcg caggaggacg aggacggcga ctacgaggag
ctggtgctag ccttgcgttc cgaggaggac ggcctggccg
aagcacccga gcacggaacc acagccacct tccaccgctg
cgccaaggat ccgtggaggt tgcctggcac ctacgtggtg
gtgctgaagg aggagaceca cctctcgcag tcagagcgca
ctgcccgccg cctgcaggcc caggctgccc gccggggata
cctcaccaag atcctgcatg tcttccatgg ccttcttcct
ggcttcctgg tgaagatgag tggcgacctg ctggagctgg
ccttgaagtt gccccatgtc gactacateg aggaggactc
ctctgtcttt gcccagagca tcccgtggaa cctggagcgg
attacccctc cacggtaccg ggcggatgaa taccagcccc
ccgacggagg cagcctggtg gaggtgtatc tcctagacac
cagcatacag agtgaccacc gggaaatcga gggcagggtc
atggtcaccg acttcgagaa tgtgcccgag gaggacggga
cccgcttcca cagacaggcc agcaagtgtg acagtcatgg
cacccacctg gcaggggtgg tcagcggccg ggatgccggc
gtggccaagg gtgccagcat gcgcagcctg cgcgtgctca
actgccaagg gaagggcacg gttagcggca ccctcatagg
cctggagttt attcggaaaa gccagctggt ccagcctgtg
gggccactgg tggtgctgct gcccctggcg ggtgggtaca
gccgcgtcct caacgccgcc tgccagcgcc tggcgagggc
tggggtcgtg ctggtcaccg ctgccggcaa cttccgggac
gatgcctgcc tctactcccc agcctcagct cccgaggtca
tcacagttgg ggccaccaat gcccaggacc agccggtgac
cctggggact ttggggacca actttggccg ctgtgtggac
ctctttgccc caggggagga catcattggt gcctccagcg
actgcagcac ctgctttgtg tcacagagtg ggacatcaca
ggctgctgcc cacgtggctg gcattgcagc catgatgctg
tctgccgagc cggagctcac cctggccgag ttgaggcaga
gactgatcca cttctctgcc aaagatgtca tcaatgaggc
ctggttccct gaggaccagc gggtactgac ccccaacctg
gtggccgccc tgccccccag cacccatggg gcaggttggc
agctgttttg caggactgtg tggtcagcac actcggggcc
tacacggatg gccacagcca tcgcccgctg cgccccagat
gaggagctgc tgagctgctc cagtttctcc aggagtggga
agcggcgggg cgagcgcatg gaggcccaag ggggcaagct
ggtctgccgg gcccacaacg cttttggggg tgagggtgtc
tacgccattg ccaggtgctg cctgctaccc caggccaact
gcagcgtcca cacagctcca ccagctgagg ccagcatggg
gacccgtgtc cactgccacc aacagggcca cgtcctcaca
ggctgcagct cccactggga ggtggaggac cttggcaccc
acaagccgcc tgtgctgagg ccacgaggtc agcccaacca
gtgcgtgggc cacagggagg ccagcatcca cgcttcctgc
tgccatgccc caggtctgga atgcaaagtc aaggagcatg
gaatcccggc ccctcaggag caggtgaccg tggcctgcga
ggagggctgg accctgactg gctgcagtgc cctccctggg
acctcccacg tcctgggggc ctacgccgta gacaacacgt
gtgtagtcag gagccgggac gtcagcacta caggcagcac
cagcgaagag gccgtgacag ccgttgccat ctgctgccgg
agccggcacc tggcgcaggc ctcccaggag ctccagtga

PCSK9 translation (SEQ I.D. 2):

MGTVSSRRSWWPLPLLLLLLLLLGPAGARAQEDEDGDYEELVLALRSEED
GLAEAPEHGTTATFHRCAKDPWRLPGTYVVVLKEETHLSQSERTARRLQA
QAARRGYLTKILHVFHGLLPGFLVKMSGDLLELALKLPHVDYIEEDSSVF
AQSIPWNLERITPPRYRADEYQPPDGGSLVEVYLLDTSIQSDHREIEGRV
MVTDFENVPEEDGTRFHRQASKCDSHGTHLAGVVSGRDAGVAKGASMRSL
RVLNCQGKGTVSGTLIGLEFIRKSQLVQPVGPLVVLLPLAGGYSRVLNAA
CQRLARAGVVLVTAAGNFRDDACLYSPASAPEVITVGATNAQDQPVTLGT
LGTNFGRCVDLFAPGEDIIGASSDCSTCFVSQSGTSQAAAHVAGIAAMML
SAEPELTLAELRQRLIHFSAKDVINEAWFPEDQRVLTPNLVAALPPSTHG
AGWQLFCRTVWSAHSGPTRMATAIARCAPDEELLSCSSFSRSGKRRGERM
EAQGGKLVCRAHNAFGGEGVYAIARCCLPQANCSVHTAPPAEASMGTRVH
CHQQGHVLTGCSSHWEVEDLGTHKPPVLRPRGQPNQCVGHREASIHASCC
HAPGLECKVKEHGIPAPQEQVTVACEEGWTLTGCSALPGTSHVLGAYAVD
NTCVVRSRDVSTTGSTSEEAVTAVAICCRSRHLAQASQELQ

Sequence of flexible linker (SEQ ID No. 5):

Aaggcgcgccatcgatggtggcggtggctctggaggtggtgggtcc

Sequence of the IFP2 reporter (SEQ ID No. 6):

AAGAACGGCATCAAGGCGAACTTCAAGATCCGCCACAACATCGAGGACGG
CGGCGTGCAGCTCGCCGACCACTACCAGCAGAACACCCCCATCGGCGACG
GCCCCGTGCTGCTGCCCGACAACCACTACCTGAGCTACCAGTCCGCCCTG
AGCAAAGACCCCAACGAGAAGCGCGATCACATGGTCCTGCTGGAGTTCGT
GACCGCCGCCGGGATCACTCTCGGCATGGACGAGCTGTACAAGTAA

Sequence analysis of the resulting PCSK9-IFP2 construct was performed to confirm correct coding sequence and in-frame fusion to the reporter. Similarly, the wild-type LDLR coding sequence was amplified from a human cDNA encoding the LDL receptor (SEQ I.D. No. 7) using the following primers: forward primer 5′-ATG GGG CCC TGG GGC TGG AAA TT (SEQ ID No. 9) and reverse primer: 5′-TCA GGA AGG GTT CTG GGC AGG G (SEQ ID No. 10) by PCR The 5′-end of the forward and reverse primers contained Not I or Asc I restriction enzyme sites, respectively, which were used to fuse the coding sequence of LDLR in-frame to the N-terminus of the IFP1 reporter fragment via a 10 amino acid flexible linker in the mammalian expression vector pcDNA3.

The sequence of the wild-type LDLR is: (SEQ I.D. No. 7)

acatttgaaa atcaccccac tgcaaactcc tccccctgct
agaaacctca cattgaaatg ctgtaaatga cgtgggcccc
gagtgcaatc gcgggaagcc agggtttcca gctaggacac
agcaggtcgt gatccgggtc gggacactgc ctggcagagg
ctgcgagcat ggggccctgg ggctggaaat tgcgctggac
cgtcgccttg ctcctcgccg cggcggggac tgcagtgggc
gacagatgcg aaagaaacga gttccagtgc caagacggga
aatgcatctc ctacaagtgg gtctgcgatg gcagcgctga
gtgccaggat ggctctgatg agtcccagga gacgtgcttg
tctgtcacct gcaaatccgg ggacttcagc tgtgggggcc
gtgtcaaccg ctgcattcct cagttctgga ggtgcgatgg
ccaagtggac tgcgacaacg gctcagacga gcaaggctgt
ccccccaaga cgtgctccca ggacgagttt cgctgccacg
atgggaagtg catctctcgg cagttcgtct gtgactcaga
ccgggactgc ttggacggct cagacgaggc ctcctgcccg
gtgctcacct gtggtcccgc cagcttccag tgcaacagct
ccacctgcat cccccagctg tgggcctgcg acaacgaccc
cgactgcgaa gatggctcgg atgagtggcc gcagcgctgt
aggggtcttt acgtgttcca aggggacagt agcccctgct
cggccttcga gttccactgc ctaagtggcg agtgcatcca
ctccagctgg cgctgtgatg gtggccccga ctgcaaggac
aaatctgacg aggaaaactg cgctgtggcc acctgtcgcc
ctgacgaatt ccagtgctct gatggaaact gcatccatgg
cagccggcag tgtgaccggg aatatgactg caaggacatg
agcgatgaag ttggctgcgt taatgtgaca ctctgcgagg
gacccaacaa gttcaagtgt cacagcggcg aatgcatcac
cctggacaaa gtctgcaaca tggctagaga ctgccgggac
tggtcagatg aacccatcaa agagtgcggg accaacgaat
gcttggacaa caacggcggc tgttcccacg tctgcaatga
ccttaagatc ggctacgagt gcctgtgccc cgacggcttc
cagctggtgg cccagcgaag atgcgaagat atcgatgagt
gtcaggatcc cgacacctgc agccagctct gcgtgaacct
ggagggtggc tacaagtgcc agtgtgagga aggcttccag
ctggaccccc acacgaaggc ctgcaaggct gtgggctcca
tcgcctacct cttcttcacc aaccggcacg aggtcaggaa
gatgacgctg gaccggagcg agtacaccag cctcatcccc
aacctgagga acgtggtcgc tctggacacg gaggtggcca
gcaatagaat ctactggtct gacctgtccc agagaatgat
ctgcagcacc cagcttgaca gagcccacgg cgtctcttcc
tatgacaccg tcatcagcag agacatccag gcccccgacg
ggctggctgt ggactggatc cacagcaaca tctactggac
cgactctgtc ctgggcactg tctctgttgc ggataccaag
ggcgtgaaga ggaaaacgtt attcagggag aacggctcca
agccaagggc catcgtggtg gatcctgttc atggcttcat
gtactggact gactggggaa ctcccgccaa gatcaagaaa
gggggcctga atggtgtgga catctactcg ctggtgactg
aaaacattca gtggcccaat ggcatcaccc tagatctcct
cagtggccgc ctctactggg ttgactccaa acttcactcc
atctcaagca tcgatgtcaa cgggggcaac cggaagacca
tcttggagga tgaaaagagg ctggcccacc ccttctcctt
ggccgtcttt gaggacaaag tattttggac agatatcatc
aacgaagcca ttttcagtgc caaccgcctc acaggttccg
atgtcaactt gttggctgaa aacctactgt ccccagagga
tatggttctc ttccacaacc tcacccagcc aagaggagtg
aactggtgtg agaggaccac cctgagcaat ggcggctgcc
agtatctgtg cctccctgcc ccgcagatca acccccactc
gcccaagttt acctgcgcct gcccggacgg catgctgctg
gccagggaca tgaggagctg cctcacagag gctgaggctg
cagtggccac ccaggagaca tccaccgtca ggctaaaggt
cagctccaca gccgtaagga cacagcacac aaccacccga
cctgttcccg acacctcccg gctgcctggg gccacccctg
ggctcaccac ggtggagata gtgacaatgt ctcaccaagc
tctgggcgac gttgctggca gaggaaatga gaagaagccc
agtagcgtga gggctctgtc cattgtcctc cccatcgtgc
tcctcgtctt cctttgcctg ggggtcttcc ttctatggaa
gaactggcgg cttaagaaca tcaacagcat caactttgac
aaccccgtct atcagaagac cacagaggat gaggtccaca
tttgccacaa ccaggacggc tacagctacc cctcgagaca
gatggtcagt ctggaggatg acgtggcgtg aacatctgcc
tggagtcccg tccctgccca gaacccttcc tgagacctcg
ccggccttgt tttattcaaa gacagagaag accaaagcat
tgcctgccag agctttgttt tatatattta ttcatctggg
aggcagaaca ggcttcggac agtgcccatg caatggcttg
ggttgggatt ttggtttctt cctttcctcg tgaaggataa
gagaaacagg cccgggggga ccaggatgac acctccattt
ctctccagga agttttgagt ttctctccac cgtgacacaa
tcctcaaaca tggaagatga aaggggaggg gatgtcaggc
ccagagaagc aagtggcttt caacacacaa cagcagatgg
caccaacggg accccctggc cctgcctcat ccaccaatct
ctaagccaaa cccctaaact caggagtcaa cgtgtttacc
tcttctatgc aagccttgct agacagccag gttagccttt
gccctgtcac ccccgaatca tgacccaccc agtgtctttc
gaggtgggtt tgtaccttcc ttaagccagg aaagggattc
atggcgtcgg aaatgatctg gctgaatccg tggtggcacc
gagaccaaac tcattcacca aatgatgcca cttcccagag
gcagagcctg agtcactggt cacccttaat atttattaag
tgcctgagac acccggttac cttggccgtg aggacacgtg
gcctgcaccc aggtgtggct gtcaggacac cagcctggtg
cccatcctcc cgacccctac ccacttccat tcccgtggtc
tccttgcact ttctcagttc agagttgtac actgtgtaca
tttggcattt gtgttattat tttgcactgt tttctgtcgt
gtgtgttggg atgggatccc aggccaggga aagcccgtgt
caatgaatgc cggggacaga gaggggcagg ttgaccggga
cttcaaagcc gtgatcgtga atatcgagaa ctgccattgt
cgtctttatg tccgcccacc tagtgcttcc acttctatgc
aaatgcctcc aagccattca cttccccaat cttgtcgttg
atgggtatgt gtttaaaaca tgcacggtga ggccgggcgc
agtggctcac gcctgtaatc ccagcacttt gggaggccga
ggcgggtgga tcatgaggtc aggagatcga gaccatcctg
gctaacacgt gaaaccccgt ctctactaaa aatacaaaaa
attagccggg cgtggtggcg ggcacctgta gtcccagcta
ctcgggaggc tgaggcagga gaatggtgtg aacccgggaa
gcggagcttg cagtgagccg agattgcgcc actgcagtcc
gcagtctggc ctgggcgaca gagcgagact ccgtctcaaa
aaaaaaaaac aaaaaaaaac catgcatggt gcatcagcag
cccatggcct ctggccaggc atggcgaggc tgaggtggga
ggatggtttg agctcaggca tttgaggctg tcgtgagcta
tgattatgcc actgctttcc agcctgggca acatagtaag
accccatctc ttaaaaaatg aatttggcca gacacaggtg
cctcacgcct gtaatcccag cactttggga ggctgagctg
gatcacttga gttcaggagt tggagaccag gcctgagcaa
caaagcgaga tcccatctct acaaaaacca aaaagttaaa
aatcagctgg gtacggtggc acgtgcctgt gatcccagct
acttgggagg ctgaggcagg aggatcgcct gagcccagga
ggtggaggtt gcagtgagcc atgatcgagc cactgcactc
cagcctgggc aacagatgaa gaccctattt cagaaataca
actataaaaa aataaataaa tcctccagtc tggatcgttt
gacgggactt caggttcttt ctgaaatcgc cgtgttactg
ttgcactgat gtccggagag acagtgacag cctccgtcag
actcccgcgt gaagatgtca caagggattg gcaattgtcc
ccagggacaa aacactgtgt cccccccagt gcagggaacc
gtgataagcc tttctggttt cggagcacgt aaatgcgtcc
ctgtacagat agtggggatt ttttgttatg tttgcacttt
gtatattggt tgaaactgtt atcacttata tatatatata
tacacacata tatataaaat ctatttattt ttgcaaaccc
tggttgctgt atttgttcag tgactattct cggggccctg
tgtagggggt tattgcctct gaaatgcctc ttctttatgt
acaaagatta tttgcacgaa ctggactgtg tgcaacgctt
tttgggagaa tgatgtcccc gttgtatgta tgagtggctt
ctgggagatg ggtgtcactt tttaaaccac tgtatagaag
gtttttgtag cctgaatgtc ttactgtgat caattaaatt
tcttaaatga accaatttgt ctaaa

LDLR translation is: (SEQ ID No. 8)

MGPWGWKLRWTVALLLAAAGTAVGDRCERNEFQCQDGKCISYKWVCDGSA
ECQDGSDESQETCLSVTCKSGDFSCGGRVNRCIPQFWRCDGQVDCDNGSD
EQGCPPKTCSQDEFRCHDGKCISRQFVCDSDRDCLDGSDEASCPVLTCGP
ASFQCNSSTCIPQLWACDNDPDCEDGSDEWPQRCRGLYVFQGDSSPCSAF
EFHCLSGECIHSSWRCDGGPDCKDKSDEENCAVATCRPDEFQCSDGNCIH
GSRQCDREYDCKDMSDEVGCVNVTLCEGPNKFKCHSGECITLDKVCNMAR
DCRDWSDEPIKECGTNECLDNNGGCSHVCNDLMGYECLCPDGFQLVAQRR
CEDIDECQDPDTCSQLCVNLEGGYKCQCEEGFQLDPHTKACKAVGSIAYL
FFTNRHEVRKMTLDRSEYTSLIPNLRNVVALDTEVASNRIYWSDLSQRMI
CSTQLDRAHGVSSYDTVISRDIQAPDGLAVDWIHSNIYWTDSVLGTVSVA
DTKGVKRKTLFRENGSKPRAIVVDPVHGFMYWTDWGTPAKIKKGGLNGVD
IYSLVTENIQWPNGITLDLLSGRLYWVDSKLHSISSIDVNGGNRKTILED
EKRLAHPFSLAVFEDKVFWTDIINEAIFSANRLTGSDVNLLAENLLSPED
MVLFHNLTQPRGVNWCERTTLSNGGCQYLCLPAPQINPHSPKFTCACPDG
MLLARDMRSCLTEAEAAVATQETSTVRLKVSSTAVRTQHTTTRPVPDTSR
LPGATPGLTTVEIVTMSHQALGDVAGRGNEKKPSSVRALSIVLPIVLLVF
LCLGVFLLWKNWRLKNINSINFDNPVYQKTTEDEVHICHNQDGYSYPSRQ
MVSLEDDVA

Sequence of the flexible linker: (SEQ ID No. 11)

Aaggcgcgccatcgatggtggcggtggctctggaggtggtgggtcc

Sequence of the IFP1 reporter: (SEQ ID No. 12)

GTGAGCAAGGGCGAGGAGCTGTTCACCGGGGTGGTGCCCATCCTGGTCGA
GCTGGACGGCGACGTAAACGGCCACAAGTTCAGCGTGTCCGGCGAGGGCG
AGGGCGATGCCACCTACGGCAAGCTGACCCTGAAGTTCATCTGCACCACC
GGCAAGCTGCCCGTGCCCTGGCCCACCCTCGTGACCACCCTCGGCTACGG
CCTGCAGTGCTTCGCCCGCTACCCCGACCACATGAAGCAGCACGACTTCT
TCAAGTCCGCCATGCCCGAAGGCTACGTCCAGGAGCGCACCATCTTCTTC
AAGGACGACGGCAACTACAAGACCCGCGCCGAGGTGAAGTTCGAGGGCGA
CACCCTGGTGAACCGCATCGAGCTGAAGGGCATCGACTTCAAGGAGGACG
GCAACATCCTGGGGCACAAGCTGGAGTACAACTACAACAGCCACAACGTC
TATATCACGGCCGACAAGCAGTAA

Sequence analysis of the resulting LDLR-IFP1 construct was performed to confirm correct coding sequence and in-frame fusion to the reporter.

### Example 2

### Transient Transfection and Imaging

HEK 293T cells were seeded in normal growth media containing DMEM and 10% FBS at 1.5×10⁴ in PDL-coated 96-well plates 24 hours prior to transfection. Cells were transfected with 50 ng of each construct DNA per well with Fugene 6, using conditions recommended by the manufacturer. Cells were allowed to express the construct pairs for 24- or 48 h, then the cells were simultaneously fixed and stained with either a 1:300 dilution of Hoescht 33342 (Molecular Probes, Eugene, Oreg.) or a 1:1000 dilution of Draq5 (Biostatus, Shepshed, Leicestershire, U.K.) in 4% formaldehyde for 15 minutes at room temperature. The cells were washed to remove fixative, and overlaid with a small volume of Hank's Buffered Salt Solution. Images were acquired on a Discovery-1 (Molecular Devices) epifluorescence microscope using the 20× objective, and DAPI and FITC filter sets. (with excitation at 350 and 488 nm wave lengths) or on an Opera (Perkin Elmer) confocal microscope using the 20× water objective with the following excitation and emission settings: Ex 488 nm/Em 535 nm (YFP) and Ex 635 nm/Em 640 nm (Draq5).

### Example 3

### PCSK9/LDLR Protein Complementation Assay that Reflects the Functional Characteristics of Endogenous PCSK9/LDLR Association, Localization and Trafficking

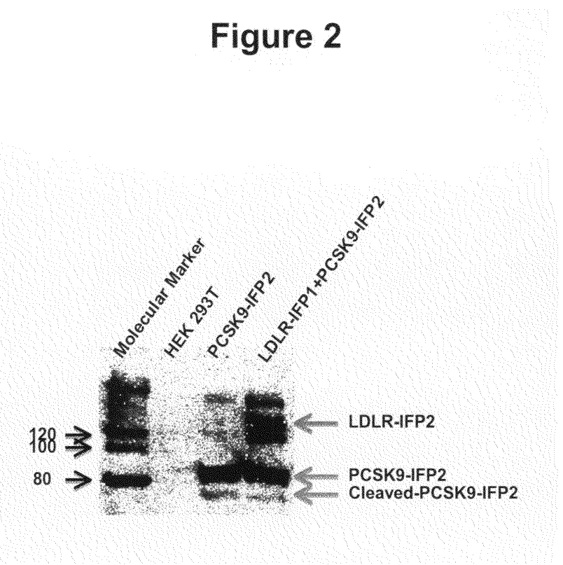

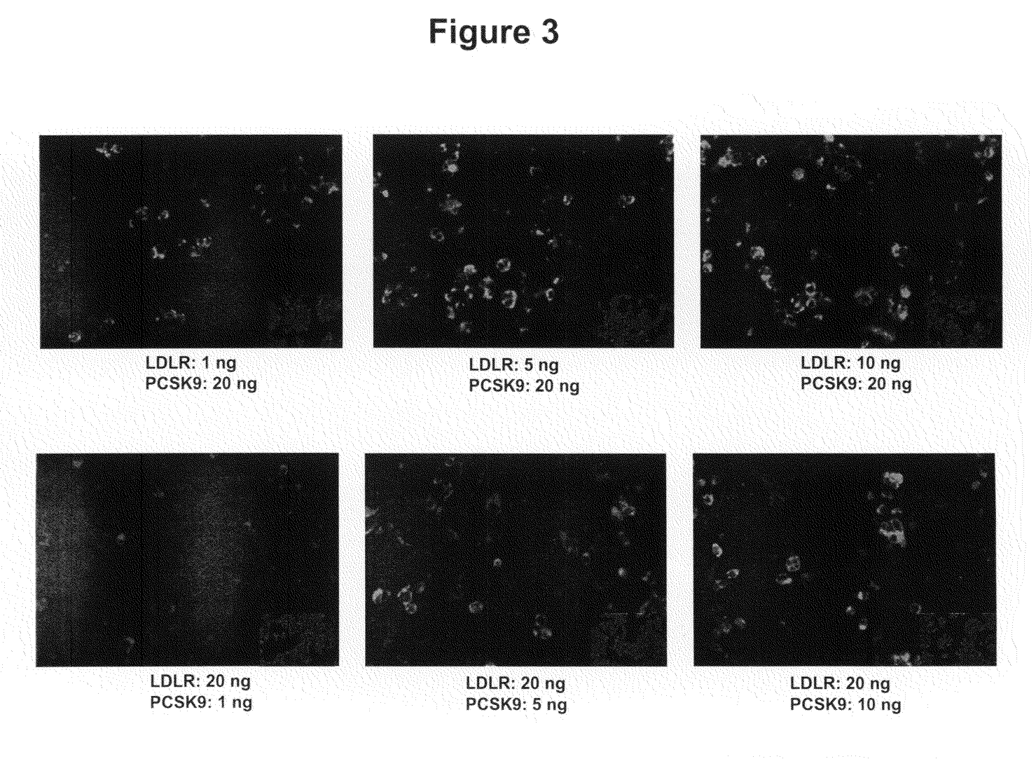

To test for in vitro PCSK9 function and activity, we developed a protein complementation assay (PCA) with PCSK9-IFP2 and the LDL-IFP1 receptor (PCSK9/LDLR). Expression of wild type PCSK9-IFP2 in transfected HEK293 cells was confirmed by Western blot using an antibody against YFP (FIG. 2). Cotranfection of HEK cells with increasing ratios of PCSK9-IFP2 and LDLR-IFP1 DNA constructs results in increased PCA signal (FIG. 3) but not when each construct is expressed alone. The intracellular localization of the wild-type PCSK9-IFP2/LDLR-IFP1 PCA interaction is similar to the known localization of wild type PCSK9 in the endoplasmic reticulum (ER) and post-ER compartments in various cell types (20, 21). The PCA signal for the PCSK9-IFP2/LDLR-IFP1 pair appears to be localized to multiple intracellular trafficking sites including the ER, endosomes and the cell surface.

### Example 4

### Effects of Known Compounds on PCSK9 PCA

In order to test the viability of using the PCSK9-IFP2/LDLR-IFP1 PCA as a drug discovery tool, we tested a drug plate containing 50 known drugs at 3 doses in the assay (Table 10) as well as several protein kinase inhibitors (representative compounds are depicted in Table 11). These compounds were chosen based on their predicted ability to affect lipid metabolism, intracellular trafficking or that have been shown to have effects on other proprotein convertases. We observed the following effects of known compounds on the PCSK9/LDLR complex:

1. Several compounds in the andrographalide family (small molecule proprotein convertase inhibitors) reduce formation of PCSK9/LDLR complexes (FIG. 5).

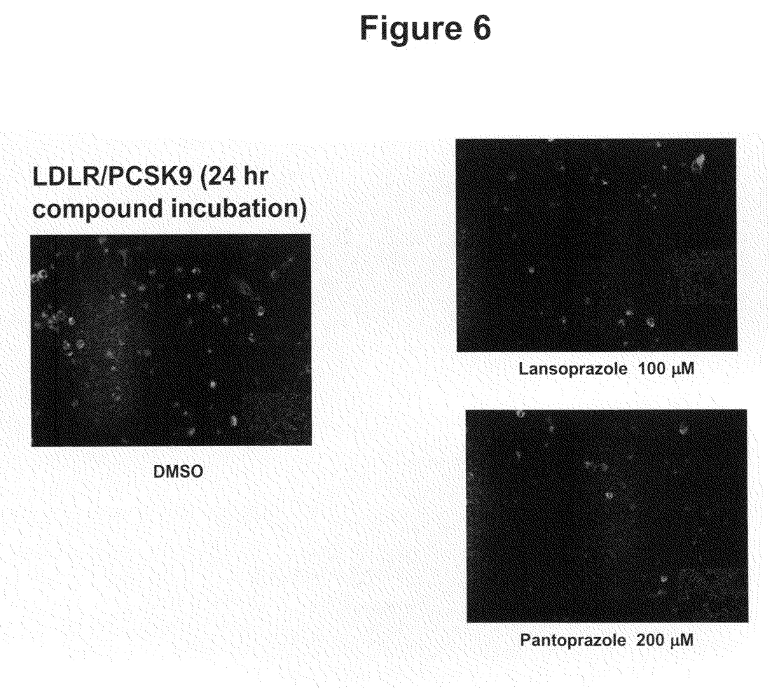

2. Lansoprazole and Pantoprazole, known H+/K+ ATPase inhibitors inhibit the formation of PCSK9/LDLR complexes (FIG. 6).

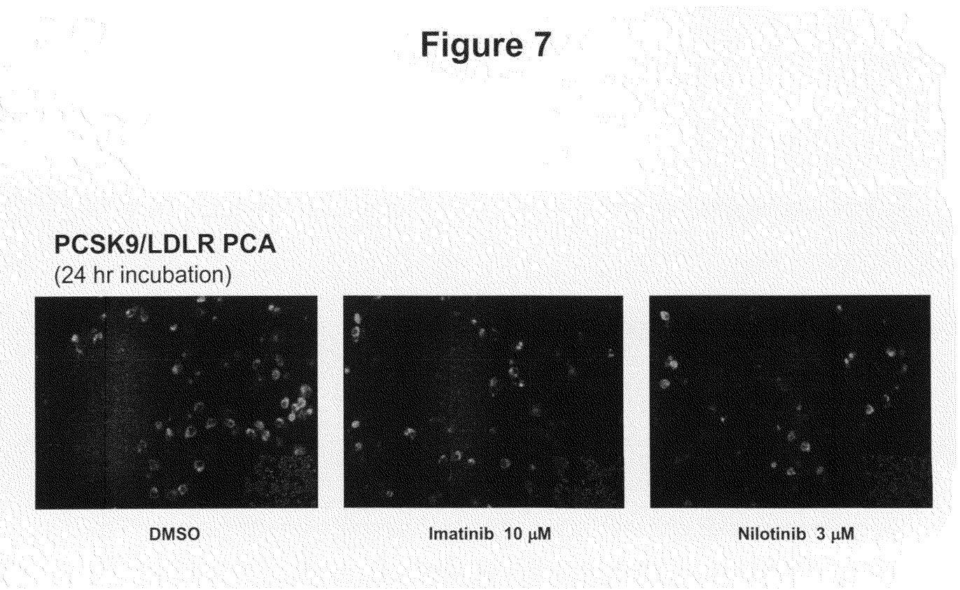

3. Imatinib and nilotinib inhibit the formation of PCSK9/LDLR complexes (FIG. 7)

The PCSK9/LDLR PCA faithfully reproduces the localization of the wild type PCSK9/LDLR protein complex. We also demonstrate that the PCSK9/LDLR PCA can identify compounds that cause an increase or decrease in activity validating this technology as a drug discovery tool. Testing of a small panel of known drugs containing compounds expected to inhibit. PCSK9/LDLR complex formation such as the andrographalides indeed resulted in a decrease in the PCA signal. Further, compounds that would be expected to increase the signal such as the statins and ACAT inhibitors induced an observable increase in the PCA signal. We also identified some surprising inhibitors of PCSK9/LDLR complex formation, including several members of the H+/K+ ATPase inhibitor family.

The invention can be extended to any assay technology that takes advantage of the PCSK9 and LDLR interaction, including but not limited to Biolumescence resonance energy transfer (BRET), Fluorescence energy transter (FRET), Homogenous time resolved fluorescence (HTRF), Scintillation proximity (SPA) and Fluoresence polarization (FP). These assays and/or the PCSK9/LDLR PCA can be used to screen compound libraries to find compounds that disrupt the PCSK9/LDLR protein complex. Compounds discovered using these methods should have functional effects on LDL uptake by cells and LDL levels in vivo as suggested by the literature.

### Example 5

A collection of small molecular weight compounds was screened. Surprisingly, we observed inhibition of the PCSK9 PCA signal by several known kinase inhibitors. To expand on this observation, we characterized the effects of a larger panel of kinase inhibitors on the PCSK9/LDLr complex. 47 compounds representing a broad range of known receptor tyrosine and serine-threonine kinase inhibitors were assessed (table 2). A subset was identified that had inhibitory effects on the PCSK9/LDLr complex (degree of assay inhibition relative to control is indicated by “% control”, Table 2).

TABLE 2
Compounds found to inhibit PCSK9/LDLr PCA

| | | Dose | % |
|---|---|---|---|
| Compounds | Purported Drug target | (μM) | Control |
| Akt Inhibitor IV | Akt | 1 | 65 |
| Akt Inhibitor IV | Akt | 3 | 40 |
| Akt Inhibitor IV | Akt | 10 | 56 |
| Gefitinib | EGFR-Her1 | 100 | 53 |
| Imatinib | BCR/Abl/PDGFR/ckit | 10 | 55 |
| Nilotinib | BCR/Abl/PDGFR/cKit | 3 | 48 |
| Nilotinib | BCR/Abl/PDGFR/cKit | 10 | 65 |
| Neratinib | ErbB1; ErbB2 | 1 | 64 |
| Neratinib | ErbB1; ErbB2 | 3 | 61 |
| Sorafenib | c-Kit; PDGF-R; Raf; | 3 | 57 |
| | VEGF-R2; VEGF | | |
| Sorafenib | c-Kit; PDGF-R; Raf; | 10 | 58 |
| | VEGF-R2; VEGF | | |
| Vandetanib | VEGFR/EGFR | 100 | 39 |
| Indirubin-3′-Monoxime | CDKs (non-selective) | 10 | 64 |
| Purvalanol A | CDKs (non-selective) | 15 | 52 |
| Roscovitine | CDKs (non-selective) | 40 | 54 |
| PI 3-K alpha Inhibitor IV | PI3K alpha | 30 | 46 |
| PI 3-Kalpha Inhibitor VIII | PI3K alpha | 1 | 44 |
| PI 3-Kalpha Inhibitor VIII | PI3K alpha | 3 | 39 |
| PI 3-Kalpha Inhibitor VIII | PI3K alpha | 10 | 29 |
| PI3K gamma/CKII inhibitor | PI3K gamma | 3 | 57 |
| PI3K gamma/CKII inhibitor | PI3K gamma | 10 | 47 |
| Wortmannin | PI3K | 1.5 | 63 |
| Src Kinase Inhibitor I | c-Src | 12.5 | 50 |
| Staurosporine | Ser/Thr kinase | 1 | 34 |
| Zotarolimus | m-TOR | 10 | 37 |
| Amitryptyline | norepinephrine receptor | 100 | 48 |
| Imipramine | serotonin/norepinephrine | 150 | 46 |
| | receptors | | |
| Clomipramine | serotonin/norepinephrine | 50 | 46 |
| | receptors | | |
| Sertraline | Serotonin receptor | 100 | 46 |
| Andrographolide | anti-inflammatory | 100 | 43 |
| Andrograpanin | anti-inflammatory | 100 | 40 |
| Cytochalasin B | Actin | 2 | 53 |
| Lomustine | DNA (cross linking) | 50 | 50 |
| Lomustine | DNA (cross linking) | 150 | 54 |
| Lanzoprazole | proton pump | 100 | 53 |
| Pantoprazole | proton pump | 200 | 36 |
| Loratidine | Histamine receptor | 30 | 59 |
| Terfenadine | Histamine receptor | 10 | 59 |
| PM20 | Cdc25A | 10 | 53 |
| Tamoxifen | Estrogen receptor | 30 | 53 |

TABLE 3
Compounds that modulate PCSK9/LDLR interactions
COMPOUND/AGENT

14-Deoxy-11,12-didehydroandrographolide
22 (R) Hydroxycholesterol
25-Hydroxycholesterol
Acetyl Podocarpic Acid Anhydride
AEBSF
ALLN
Andrograpanin
Andrographolide
Antipain
Atorvastatin
Bortezomib
Brefeldin A
Cathepsin/subtilisin inhibitor
CAY10487
Cerivastatin
Chymostatin
CI-976
Clofibrate
Colchicine
Combretastatin A-4
Docetaxel
E-64
Ecotin
EST
Ezetimibe
Fasudil
Fenofibrate
Griseofulvin
H 1152
H-89
Hesperetin
Losartan
Lovastatin
Mevastatin
Myoseverin B
Neoandrographolide
Nocodazole
Pepstatin A
Pepstatin A Methyl Ester
Protease Inhibitor Cocktail Set III
Protease Inhibitor Cocktail Set V
Pyripyropene A
Sandoz 58-035
Taxol
Tubulin Polymerization Inhibitor
Tubulin Polymerization Inhibitor II
Vinblastine
Vincristine
Y-27632
YIC-C8-434

### Example 6

### Kinase Inhibitors Increase LDL Uptake in HEPG2 Cells

We assessED whether compounds that inhibit the PCSK9/LDLr assay would have activity on LDL uptake and metabolism in a relevant cell type. PCSK9 is thought to be a negative regulator of LDLr. Thus, we predicted that compounds inhibiting the PCSK9 PCA signal would increase LDL receptor expression and LDL uptake. An assay using human LDL conjugated to DyLight™ 549 as a fluorescent probe for detection of LDL uptake into cultured human hepatocytes. A separate assay, using an LDL receptor-specific polyclonal antibody and a DyLight™ 488-conjugated secondary antibody, was also performed to localize LDL receptors

The treatment of the human hepatoma cell line HepG2 with the small molecules Imatinib (10 uM), Neratinib (10 μM), Lapatinib (10 μM) and Src Kinase inhibitor I (10 μM) resulted in a dramatic increase in LDL uptake. Quantitation of the increased LDL uptake of these and other kinase inhibitors targeting Akt, mTOR, p38, GSK3 and VEGF is shown in Table 4.

TABLE 4
Regulators of hepatic LDL uptake

| | Purported Drug | | x-fold |
|---|---|---|---|
| Compounds | target | Dose (□M) | increase |
| Akt Inhibitor X | Akt | 1 | 1.4 |
| Akt Inhibitor X | Akt | 3 | 1.7 |
| KRIBB3 | PKC delta | 1 | 1.3 |
| KRIBB3 | PKC delta | 3 | 1.6 |
| LY 303511 | mTOR | 3 | 1.4 |
| LY 303511 | mTOR | 30 | 2.1 |
| SB 202190 | p38 | 1 | 2.7 |
| SB 203580 | p38 | 3 | 3.0 |
| SB 415286 | GSK3 | 25 | 1.6 |
| Src Kinase Inhibitor I | c-src | 3 | 2.3 |
| Src Kinase Inhibitor I | c-src | 10 | 5.1 |
| PD-153035 | EGFR | 0.2 | 1.3 |
| PD-158780 | EGFR | 0.3 | 1.3 |
| Vandetanib | VEGFR/EGFR | 1 | 1.5 |
| Vandetanib | VEGFR/EGFR | 3 | 2.0 |
| Imatinib | BCR/Abl/PDGFR/cKit | 3 | 2.4 |
| Imatinib | BCR/Abl/PDGFR/cKit | 10 | 5.4 |
| Neratinib | ERBB1/ERBB2 | 10 | 2.2 |
| Lapatinib | EGFR/ERBB2 | 10 | 1.8 |
| Lapatinib | EGFR/ERBB2 | 30 | 1.9 |
| Oncostatin M (OSM) | Gp130/OSMR/LIFR | 30 ng/ml | 4.2 |

We also found that the LDL internalized following treatment with these compounds is co-localized with the LDR receptor.

TABLE 5
Several tyrosine kinase inhibitors
increase LDL uptake in Hepg2 cells

| | | Dose | % |
|---|---|---|---|
| Compounds | Drug target | (μM) | Control |
| Akt Inhibitor IV | Akt | 1 | 65 |
| Akt Inhibitor IV | Akt | 3 | 40 |
| Imatinib | BCR/Abl; PDGFR; ckit | 10 | 55 |
| Nilotinib | BCR/Abl; PDGFR; cKit | 3 | 48 |
| Nilotinib | BCR/Abl; PDGFR; cKit | 10 | 65 |
| Neratinib | ErbB1; ErbB2 | 1 | 64 |
| Neratinib | ErbB1; ErbB2 | 3 | 61 |
| Sorafenib | c-Kit; PDGFR; Raf; | 3 | 57 |
| | VEGFR2 | | |
| Sorafenib | c-Kit; PDGFR; Raf; | 10 | 58 |
| | VEGFR2 | | |
| Vandetanib | VEGFR; EGFR | 100 | 39 |
| Indirubin-3′-Monoxime | CDKs (non-selective) | 10 | 64 |
| Purvalanol A | CDKs (non-selective) | 15 | 52 |
| Roscovitine | CDKs (non-selective) | 40 | 54 |
| PI 3-Kalpha Inhibitor VIII | PI3K alpha | 1 | 44 |
| PI 3-Kalpha Inhibitor VIII | PI3K alpha | 3 | 39 |
| PI3K gamma/CKII inhibitor | PI3K gamma | 3 | 57 |
| Wortmannin | PI3K | 1.5 | 63 |
| Src Kinase Inhibitor I | c-Src | 12.5 | 50 |
| Zotarolimus | m-TOR | 10 | 37 |

TABLE 6
Several tyrosine kinase inhibitors
increase LDL uptake in Hepg2 cells

| | | Dose | x-fold |
|---|---|---|---|
| Compounds | Drug target | (μM) | increase |
| Akt Inhibitor X | Akt | 1 | 1.4 |
| Akt Inhibitor X | Akt | 3 | 1.7 |
| KRIBB3 | PKC delta | 1 | 1.3 |
| KRIBB3 | PKC delta | 3 | 1.6 |
| LY 303511 | mTOR | 3 | 1.4 |
| LY 303511 | mTOR | 30 | 2.1 |
| SB 202190 | p38 | 1 | 2.7 |
| SB 203580 | p38 | 3 | 3.0 |
| SB 415286 | GSK3 | 25 | 1.6 |
| Src Kinase Inhibitor I | c-src | 3 | 2.3 |
| Src Kinase Inhibitor I | c-src | 10 | 5.1 |
| PD-153035 | EGFR | 0.2 | 1.3 |
| PD-158780 | EGFR | 0.3 | 1.3 |
| Vandetanib | VEGFR; EGFR | 1 | 1.5 |
| Vandetanib | VEGFR; EGFR | 3 | 2.0 |
| Imatinib | BCR/Abl; PDGFR; | 3 | 2.4 |
| | ckit | | |
| Imatinib | BCR/Abl; PDGFR; | 10 | 5.4 |
| | ckit | | |
| Neratinib | ErbB1; ErbB2 | 10 | 2.2 |
| Lapatinib | EGFR; ERbB2 | 10 | 1.8 |
| Lapatinib | EGFR; ERbB2 | 30 | 1.9 |

Akt inhibitor IV: 5-(2-Benzothiazolyl)-3-ethyl-2-[2-(methylphenylamino)ethenyl]-1-phenyl-1H-benzimidazolium iodide
Akt inhibitor X: 10-(4′-(N-diethylamino)butyl)-2-chlorophenoxazine, HCl
PI3K inhibitor IV: 3-(4-Morpholinothieno[3,2-d]pyrimidin-2-yl)phenol
PI3K inhibitor VIII: N-((1E)-(6-Bromoimidazo[1,2-a]pyridin-3-yl)methylene)-Nprime-methyl-Ndoubleprime-(2-methyl-5-nitrobenzene)sulfonohydrazide
PI3K-gamma/CKII: (5-(4-Fluoro-2-hydroxyphenyl)furan-2-ylmethylene)thiazolidine-2,4-dione
Src kinase inhibitor I: 4-(4-prime-Phenoxyanilino)-6,7-dimethoxyquinazoline
Akt inhibitor IV: 5-(2-Benzothiazolyl)-3-ethyl-2-[2-(methylphenylamino)ethenyl]-1-phenyl-1H-benzimidazolium iodide
PI3K inhibitor VIII: N-((1E)-(6-Bromoimidazo[1,2-a]pyridin-3-yl)methylene)-Nprime-methyl-Ndoubleprime-(2-methyl-5-nitrobenzene)sulfonohydrazide
PI3K-gamma/CKII inhibitor: (5-(4-Fluoro-2-hydroxyphenyl)furan-2-ylmethylene)thiazolidine-2,4-dione
Src kinase inhibitor I: 4-(4-prime-Phenoxyanilino)-6,7-dimethoxyquinazoline
Akt inhibitor X: 10-(4′-(N-diethylamino)butyl)-2-chlorophenoxazine, HCl

The following patents, published patent applications as well as all their foreign counter-parts, journal articles and all cited references in each of those patents and journal articles cited therein are incorporated in their entirety by reference herein as if those references were denoted in the text:
1. US 20040161787 Protein fragment complementation assays for high-throughput and high-content screening
2. US 20040137528 Fragments of fluorescent proteins for protein fragment complementation assays
3. US 20040038298 Protein fragment complementation assays for the detection of biological or drug interactions
4. US 20030108869 Protein fragment complementation assay (PCA) for the detection of protein-protein, protein-small molecule and protein nucleic acid interactions based on the E. coli TEM-1 beta-lactamase
5. US 20030049688 Protein fragment complementation assays for the detection of biological or drug interactions
6. US 20020064769 Dynamic visualization of expressed gene networks in living cells
7. US 20010047526 Mapping molecular interactions in plants with protein fragments complementation assays
8. U.S. Pat. No. 6,428,951 Protein fragment complementation assays for the detection of biological or drug interactions
9. U.S. Pat. No. 6,294,330 Protein fragment complementation assays for the detection of biological or drug interactions
10. U.S. Pat. No. 6,270,964 Protein fragment complementation assays for the detection of biological or drug interactions
11. Abifadel M, Varret M, Rabes J P, Allard D, Ouguerram K, Devillers M, Cruaud C, Benjannet S, Wickham L, Erlich D, Derre A, Villeger L; Farnier M, Beucler I, Bruckert E, Chambaz J, Chanu B, Lecerf J M, Luc G, Moulin P, Weissenbach J, Prat A, Krempf M, Junien C, Seidah N G, Bioleau C. Mutations in PCSK9 cause autosomal dominant hypercholesterolemia. Nat. Genet. 2003; 34:154-156
12. Leren T P. Mutations in the PCSK9 gene in a Norwegian sugjects with autosomal dominant hypercholesterolemia. Clin. Genet. 2004; 65:419-422.
13. Timms K M, Wagner S, Samuels M E, Forbey K, Goldfine H, Jammulapati S, Skolnick M H, Hopkins P N, Hunt S C, Shattuck D M. A mutation in PCSK9 causing autosomal dominant hypercholesterolemia in a Utah pedigree. Hum. Genet. 2004; 114:349-353.
14. Maxwell K N, Soccio R E, Ducan E M, Sehayek E, Breslow J L. Novel putative SREBP and LXR target genes identified by microarray analysis in the liver of cholesterol-fed mice. J. Lipid Res. 2203; 44:2109-2119.
15. Horton J D, Shah N A, Warrington J A, Anderson M M, Park S W, Brown M S, Goldstein J L. Combined analysis of oligonucleotide microarray data from transgenic and knockout mice identifies direct SREBP target genes. Proc. Natl. Acad. Sci. USA. 2003; 100:12027-12032.
16. Cohen J, Pertsemlidis A, Kotowski I K, Graham R, Garcia C K, Hobbs H H. Low LDL cholesterol in individuals of African descent resulting from frequent nonsense mutations in PCSK9. Nat. Genet. 2005; 37:161-165.
17. Kotowski I K, Pertsemlidis A, Luke A, Cooper R S, Vega G L, Cohen J C, Hobbs H H. A spectrum of PCSK9 allels contributes to plasma levels of low-density lipoprotein cholesterol. Am. J. Hum. Genet. 2006; 78:410-422.
18. Berge K E, Ose L, Leren T P. Missense mutations in the PCSK9 gene are associated with hypocholesterolemia and possible increased response to statin therapy. Arterioscler. Thromb. Vasc. Biol. 2006; 26:1094-1100.
19. Cohen J C, Boerwinkle E, Mosley T H, Hobbs H H. Sequence variation in PCSK9, low LDL, and protection against coronary heart disease. N. Engl. J. Med. 2006; 354:34-42.
20. Seidah N G, Benjannet S, Wickham L, Macinkiewicz J, Jasmin S B, Stifani S, Basak A, Prat A, Chretien M. The secretory proprotein convertase neural apoptosis-regulated convertase-1 (NARC-1): liver regeneration and neuronal differentiation. Proc. Natl. Acad. Sci. USA. 2003; 100:928-933.
21. Naureckiene S, Ma L, Sreekumar K, Puradare U, Lo C F, Huang Y, Chiang L W, Grenier J M, Ozenberger B A, Jacobsen J S, Kennedy J D, Distefano P S, Wood A, Bingham B. Functional characterization of Narcl, a novel proteinases related to proteinase K. Arch. Biochem. Biophys. 2003; 420:55-67.
22. P domain paper . . .
23. Hampton E N, Knuth M W, Jun Li, Harris J L, Lesley S A, Spraggon G. The self-inhibited structure of the full-length PCSK9 at 1.9 Å reveals structural homology with resistin within the C-terminal domain. Proc. Natl. Acad. Sci. USA. 2007; 104:14604-14609.
24. Maxwell K, Berslow J L. Adenoviral-mediated expression of PCSK9 in mice results in a low-density lipoprotein receptor knockout phenotype. Proc. Natl. Acad. Sci. 2004; 101:7100-7105.
25. Rashid S, Curtis D E, Garuti R, Anderson N N, Bashmakov Y, Ho Y. K., Hammer R E, Moon Y-A, Horton J D. Decreased plasma cholesterol and hypersensitivity to statins in mice lacking PCSK9. Proc. Natl. Acad. Sci. 2005; 102:5374-5379.
26. Clinical data presentation from Alnylam pharmaceuticals hypercholesterolemia program at the XVI International Symposium on Drugs Affecting Lipid Metabolism held in New York City, Oct. 4-7, 2007
27. McNutt M C, Lagace T A, Horton J A. Catalytic activity is not required for secreted PCSK9 to reduce LDL receptors in HepG2 cells. J. Biol. Chem. 2007; 282:20799-20803
28. Li J, Tumanut C, Gavigan J-A, Huang W-J, Hampton E N, Tumanut R, Suen K F, Trauger J W, Spraggon G, Lesley S A, Liau G, Yowe D, Harris J L. Secreted PCSK9 promotes LDL receptor degradation inidependently of proteolytic activity. Biochem. J. 2007; 406:203-207.
29. Zhao Z, Tuakli-Wosomu Y, Lagace T, Kinch L, Grishin N V, Horton J D, Cohen J C, Hobbs H H. Molecular Characterization of loss-of-function mutations in PCSK9 and identification of a compound heterozygote. Am. J. Hum. Genet. 2006; 79:514-523.
30. Nassoury N, Blasiole D A, Tebon Oler A, Benjannet S, Hamelin J, Poupon V, McPherson P S, Attie A D, Prat A, Seidah N G. The cellular trafficking of the secretory proprotein convertase pcsk9 and its dependence on the LDLR. Traffic 2007; 8:718-732.
31. Poirier S, Mayer G, Benjannet S, Bergeron E, Marcinkiewicz J, Narroury N, Mayer H, NimpfJ, Prat A, Seidah N G. The lipoprotein convertase PCSK9 induces the degradation of LDLR and its closest family members VLDLR and APOER2. J. Biol. Chem. in press. Published Nov. 26, 2007 as Manuscript M708098200.
32. Fan D, Yancey P G, Qiu S, Ding L, Weeber E J, Linton M F, Fazio S. Self-association of human PCSK9 correlates with its LDLR-Degrading activity. Biochemistry. 2008 Jan. 16; [Epub ahead of print]

While the many embodiments of the invention have been disclosed above and include presently preferred embodiments, many other embodiments and variations are possible within the scope of the present disclosure and in the appended claims that follow. Accordingly, the details of the preferred embodiments and examples provided are not to be construed as limiting. It is to be understood that the terms used herein are merely descriptive rather than limiting and that various changes, numerous equivalents may be made without departing from the spirit or scope of the claimed invention.

We claim:

1. A method of assaying protein-protein interactions associated with proteins involved in lipid pathways using a protein fragment complementation assays, said method comprising the steps of:
(a) identifying protein molecules that interact with said protein associated with lipid pathways;
(b) selecting a protein reporter molecule;
(c) effecting fragmentation of said protein reporter molecule such that said fragmentation results in reversible loss of reporter function;
(d) fusing or attaching fragments of said protein reporter molecule separately to said interacting protein molecules as defined in step (a);
(e) transfecting cells with nucleic acid constructs coding for the products of step (d);
(f) reassociating said reporter fragments through interactions of the protein molecules that are fused or attached to said fragments; and
(g) measuring directly or indirectly the activity of said reporter molecule resulting from the reassociation of said reporter fragments.

2. A method of assaying protein-protein interactions associated with the Proprotein convertase subtilisin kexin 9 (PCSK9) using a protein fragment complementation assays, said method comprising the steps of:
(a) identifying protein molecules that interact with said PCSK9 protein;
(b) selecting a protein reporter molecule;
(c) effecting fragmentation of said protein reporter molecule such that said fragmentation results in reversible loss of reporter function;
(d) fusing or attaching fragments of said protein reporter molecule separately to said interacting protein molecules as defined in step (a);
(e) transfecting cells with nucleic acid constructs coding for the products of step (d);
(f) reassociating said reporter fragments through interactions of the protein molecules that are fused or attached to said fragments; and
(g) measuring directly or indirectly the activity of said reporter molecule resulting from the reassociation of said reporter fragments.

3. The method of claim 2, wherein said interacting proteins (a) are low density lipoprotein receptor proteins (LDLR).

4. The method of claim 2, wherein said protein reporter molecule is selected from the group consisting of enzymes and fluorescent proteins.

5. The method of claim 4, wherein said enzyme reporter molecules are selected from the group consisting of dihydrofolate reductase, luciferase, β-lactamase, neomycin phospho-transferase and hygromycin phospho-transferase.

6. The method of claim 4, wherein said fluorescent protein reporter molecules are selected from the group consisting green fluorescent protein, mutants of green fluorescent proteins, yellow fluorescent proteins, mutants of yellow fluorescent proteins, red fluorescent protein, and mutants of red fluorescent protein.

7. A method of screening a candidate drug, a compound library or a biological extract to identify activators or inhibitors of protein-protein interactions associated with the Proprotein convertase subtilisin kexin 9 (PCSK9) protein using protein complementation assays, said method comprising the steps of:
(a) selecting a protein reporter molecule;
(b) effecting fragmentation of said protein reporter molecule such that said fragmentation results in reversible loss of reporter function;
(c) fusing or attaching fragments of said protein reporter molecule separately to the PCSK9 protein and other protein molecules known to have an interaction with said PCSK9 protein;
(d) transfecting cells with nucleic acid constructs coding for the products of step (C);
(e) testing the effects of said candidate drug, compound library, or biological extract on the protein interaction of interest by contacting said cells as defined in step (D) with said candidate drug, compound library or biological extract; and
(f) measuring and/or detecting directly or indirectly the activity resulting from the reassociation of the reporter fragments which had been fused to the interacting proteins, to identify specific agents that activate or inhibit the interaction of interest.

8. The method of claim 7, wherein said other known interacting proteins (C) are low density lipoprotein receptor proteins (LDLR).

9. The method of claim 7, wherein said protein reporter molecule is selected from the group consisting of enzymes and fluorescent proteins.

10. The method of claim 7, wherein said enzyme reporter molecules are selected from the group consisting of dihydrofolate reductase, luciferase, β-lactamase, neomycin phospho-transferase and hygromycin phospho-transferase.

11. The method of claim 7, wherein said fluorescent protein reporter molecules are selected from the group consisting green fluorescent protein, mutants of green fluorescent proteins, yellow fluorescent proteins, mutants of yellow fluorescent proteins, red fluorescent protein, and mutants of red fluorescent protein.

12. A method for identifying a drug lead that modulates the activity of protein-protein interactions between a first protein and a second protein, said first and second proteins being associated with lipid regulating pathways using protein complementation assays, said method comprising the steps of:
(a) assembling a collection or a library of compounds, said collection or library selected from the group consisting of candidate drugs, natural products, chemical compounds and/or biological extracts;
(b) selecting a protein reporter molecule;
(c) effecting fragmentation of said protein reporter molecule such that said fragmentation results in reversible loss of reporter function;
(d) fusing or attaching fragments of said protein reporter molecule separately to said first protein and second protein associated with lipid regulating pathways;
(e) transfecting cells with nucleic acid constructs coding for the products of step (d);
(f) screening said collection or library by contacting said cells as defined in (e) with one or more test elements from said collection or library; and
(g) detecting directly or indirectly the activity resulting from the reassociation of the reporter fragments which had been fused to the interacting proteins, one or more properties of said assay; wherein a change in one or more properties of said assay in the presence of any of said test elements, relative to the absence of said test element, is used to identify a drug lead that modulates a protein-protein interaction associated with lipid regulating pathways.

13. A method for identifying a drug lead that modulates the activity of protein-protein interactions between the PCSK9 protein and the LDLR protein using protein complementation assays, said method comprising the steps of:
(a) assembling a collection or a library of compounds, said collection or library selected from the group consisting of candidate drugs, natural products, chemical compounds and/or biological extracts;
(b) selecting a protein reporter molecule;
(c) effecting fragmentation of said protein reporter molecule such that said fragmentation results in reversible loss of reporter function;
(d) fusing or attaching fragments of said protein reporter molecule separately to said interacting PCSK9 protein and the LDLR protein;
(e) transfecting cells with nucleic acid constructs coding for the products of step (d);
(f) screening said collection or library by contacting said cells as defined in (e) with one or more test elements from said collection or library; and
(g) detecting directly or indirectly the activity resulting from the reassociation of the reporter fragments which had been fused to the interacting proteins, one or more properties of said assay; wherein a change in one or more properties of said assay in the presence of any of said test elements, relative to the absence of said test element, is used to identify a drug lead that modulates a PCSK9-LDLR interaction.

14. A method for treating dislipidemias which method comprises administering to a patient in need thereof an effective amount of receptor and non-receptor tyrosine kinase inhibitors.

15. A method for treating dislipidemias in a patient in need thereof, which method comprises modulating protein-protein interactions associated with lipid regulating pathways by administering to said patient effective amounts of receptor and non-receptor tyrosine kinase inhibitors.


SEQUENCE LISTING (12 sequences)

SEQ ID NO: 1 - DNA, 2079 bp, Mus musculus
  wild-type proprotein convertase subtilisin/kexin type 9 (1)..(2079)
atgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtgtggtcagcacactcggggcctacacggatggccacagccatcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaagaggccgtgacagccgttgccatctgctgccggagccggcacctggcgcaggcctcccaggagctccagtga

SEQ ID NO: 2 - protein, 692 aa, Mus musculus
  wild-type proprotein convertase subtilisin/kexin type 9 (1)..(692)
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEEAVTAVAICCRSRHLAQASQELQ

SEQ ID NO: 3 - DNA, 42 bp, artificial
  Synthetic
ataagaatgcggccgcaccatgggcaccgtcagctccaggcg

SEQ ID NO: 4 - DNA, 31 bp, artificial
  synthetic
ggcgcgcccctggagctcctgggaggcctgc

SEQ ID NO: 5 - DNA, 46 bp, artificial
  synthesized
aaggcgcgccatcgatggtggcggtggctctggaggtggtgggtcc

SEQ ID NO: 6 - DNA, 246 bp, artificial
  synthesized
aagaacggcatcaaggcgaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 7 - DNA, 5265 bp, Mus musculus
  low density lipoprotein receptor (1)..(5265)
acatttgaaaatcaccccactgcaaactcctccccctgctagaaacctcacattgaaatgctgtaaatgacgtgggccccgagtgcaatcgcgggaagccagggtttccagctaggacacagcaggtcgtgatccgggtcgggacactgcctggcagaggctgcgagcatggggccctggggctggaaattgcgctggaccgtcgccttgctcctcgccgcggcggggactgcagtgggcgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccgacctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaacatctgcctggagtcccgtccctgcccagaacccttcctgagacctcgccggccttgttttattcaaagacagagaagaccaaagcattgcctgccagagctttgttttatatatttattcatctgggaggcagaacaggcttcggacagtgcccatgcaatggcttgggttgggattttggtttcttcctttcctcgtgaaggataagagaaacaggcccggggggaccaggatgacacctccatttctctccaggaagttttgagtttctctccaccgtgacacaatcctcaaacatggaagatgaaaggggaggggatgtcaggcccagagaagcaagtggctttcaacacacaacagcagatggcaccaacgggaccccctggccctgcctcatccaccaatctctaagccaaacccctaaactcaggagtcaacgtgtttacctcttctatgcaagccttgctagacagccaggttagcctttgccctgtcacccccgaatcatgacccacccagtgtctttcgaggtgggtttgtaccttccttaagccaggaaagggattcatggcgtcggaaatgatctggctgaatccgtggtggcaccgagaccaaactcattcaccaaatgatgccacttcccagaggcagagcctgagtcactggtcacccttaatatttattaagtgcctgagacacccggttaccttggccgtgaggacacgtggcctgcacccaggtgtggctgtcaggacaccagcctggtgcccatcctcccgacccctacccacttccattcccgtggtctccttgcactttctcagttcagagttgtacactgtgtacatttggcatttgtgttattattttgcactgttttctgtcgtgtgtgttgggatgggatcccaggccagggaaagcccgtgtcaatgaatgccggggacagagaggggcaggttgaccgggacttcaaagccgtgatcgtgaatatcgagaactgccattgtcgtctttatgtccgcccacctagtgcttccacttctatgcaaatgcctccaagccattcacttccccaatcttgtcgttgatgggtatgtgtttaaaacatgcacggtgaggccgggcgcagtggctcacgcctgtaatcccagcactttgggaggccgaggcgggtggatcatgaggtcaggagatcgagaccatcctggctaacacgtgaaaccccgtctctactaaaaatacaaaaaattagccgggcgtggtggcgggcacctgtagtcccagctactcgggaggctgaggcaggagaatggtgtgaacccgggaagcggagcttgcagtgagccgagattgcgccactgcagtccgcagtctggcctgggcgacagagcgagactccgtctcaaaaaaaaaaaacaaaaaaaaaccatgcatggtgcatcagcagcccatggcctctggccaggcatggcgaggctgaggtgggaggatggtttgagctcaggcatttgaggctgtcgtgagctatgattatgccactgctttccagcctgggcaacatagtaagaccccatctcttaaaaaatgaatttggccagacacaggtgcctcacgcctgtaatcccagcactttgggaggctgagctggatcacttgagttcaggagttggagaccaggcctgagcaacaaagcgagatcccatctctacaaaaaccaaaaagttaaaaatcagctgggtacggtggcacgtgcctgtgatcccagctacttgggaggctgaggcaggaggatcgcctgagcccaggaggtggaggttgcagtgagccatgatcgagccactgcactccagcctgggcaacagatgaagaccctatttcagaaatacaactataaaaaaataaataaatcctccagtctggatcgtttgacgggacttcaggttctttctgaaatcgccgtgttactgttgcactgatgtccggagagacagtgacagcctccgtcagactcccgcgtgaagatgtcacaagggattggcaattgtccccagggacaaaacactgtgtcccccccagtgcagggaaccgtgataagcctttctggtttcggagcacgtaaatgcgtccctgtacagatagtggggattttttgttatgtttgcactttgtatattggttgaaactgttatcacttatatatatatatatacacacatatatataaaatctatttatttttgcaaaccctggttgctgtatttgttcagtgactattctcggggccctgtgtagggggttattgcctctgaaatgcctcttctttatgtacaaagattatttgcacgaactggactgtgtgcaacgctttttgggagaatgatgtccccgttgtatgtatgagtggcttctgggagatgggtgtcactttttaaaccactgtatagaaggtttttgtagcctgaatgtcttactgtgatcaattaaatttcttaaatgaaccaatttgtctaaa

SEQ ID NO: 8 - protein, 860 aa, Mus musculus [sequence omitted]

SEQ ID NO: 9 - DNA, 23 bp, artificial
  synthesized
atggggccctggggctggaaatt

SEQ ID NO: 10 - DNA, 22 bp, artificial
  synthesized
tcaggaagggttctgggcaggg

SEQ ID NO: 11 - DNA, 46 bp, artificial; identical to SEQ ID NO: 5
  synthesized

SEQ ID NO: 12 - DNA, 474 bp, artificial
  synthesized
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcacggccgacaagcagtaa